(19) United States
(12) Patent Application Publication
(10) Pub. No.: US 2023/0180754 A1
(43) Pub. Date: Jun. 15, 2023

(54) HETEROCYCLIC COMPOUNDS FOR THE CONTROL OF INVERTEBRATE PESTS

(71) Applicant: BASF SE, Ludwigshafen (DE)

(72) Inventors: Nikolas Huwyler, Ludwigshafen (DE); Arun Narine, Ludwigshafen (DE); Karsten Koerber, Ludwigshafen (DE); Erik Gilberg, Ludwigshafen (DE); Joachim Dickhaut, Ludwigshafen (DE); Jan Klaas Lohmann, Ludwigshafen (DE)

(21) Appl. No.: 17/924,256

(22) PCT Filed: May 3, 2021
(86) PCT No.: PCT/EP2021/061497

§ 371 Date: Nov. 9, 2022

(30) Foreign Application Priority Data
May 13, 2020 (EP) 20174438.0
May 13, 2020 (EP) 20174439.8

(51) Int. Cl. A01N 43/653; A01P 7/04; C07D 403/04; C07D 413/14; A01N 43/80; C07D 498/04; A01N 43/90; C07D 403/14; C07D 417/14; C07D 471/04

(52) U.S. Cl. CPC A01N 43/653; A01P 7/04; C07D 403/04; C07D 413/14; A01N 43/80; C07D 498/04; A01N 43/90; C07D 403/14; C07D 417/14; C07D 471/04

(57) ABSTRACT

The invention relates to compounds of formula (I) wherein the variables have the meanings as defined in the specification, to compositions comprising them, to active compound combinations comprising them, and to their use for protecting growing plants and animals from attack or infestation by invertebrate pests, furthermore, to seed comprising such compounds.

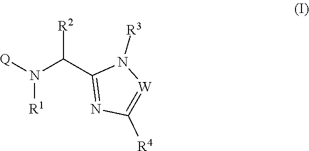

The invention relates to compounds of formula I

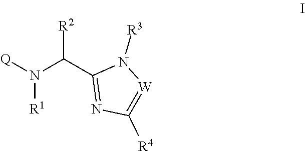

wherein
R¹ is H, OH, C₁-C₆-alkyl, C₁-C₆-haloalkyl, C₃-C₆-cycloalkyl, C₃-C₆-halocycloalkyl, C₁-C₅-alkoxy, C₁-C₄-alkyl-C₃-C₆-cycloalkyl, C₁-C₄-alkyl-C₃-C₆-halocycloalkyl, which groups are unsubstituted, or partially or fully substituted with R¹¹; or C(═N—R¹¹)R¹², C(O)R^{11a};
   R¹¹ is CN, C(O)NR¹²R¹³, C(S)NR¹²R¹³, C(O)OR¹⁴, OR¹⁴, Si(CH₃)₃; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, 5- or 6-membered hetaryl, or phenyl, which rings are unsubstituted or substituted with halogen, C₁-C₃-haloalkyl, and/or CN;
   R^{11a} is C(O)NR¹²R¹³, C(S)NR¹²R¹³, C(O)OR¹⁴, NR¹²R¹³, OR¹⁴, C₁-C₅-alkyl, C₁-C₅-haloalkyl; C₂-C₅-alkenyl; C₂-C₅-haloalkenyl; C₂-C₅-alkynyl; C₂-C₅-haloalkynyl; C₁-C₄-alkoxy-C₁-C₂-alkyl; C₃-C₄-cycloalkyl-C₁-C₂-alkyl, which ring is unsubstituted or substituted with 1 or 2 halogen; 3- to 6-membered heterocyclyl which rings are unsubstituted or substituted with halogen, C₁-C₃-haloalkyl, and/or CN;
   R¹², R¹³ are independently from each other H, C₁-C₄-alkyl, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl;
   m is 0, 1, or 2;
   R¹⁴ is H, C₁-C₄-alkyl, C₁-C₄-haloalkyl, C₃-C₆-cycloalkyl, C₃-C₄-cycloalkyl-C₁-C₂-alkyl, C₃-C₄-halocycloalkyl-C₁-C₂-alkyl, C(O)—C₁-C₄-alkyl, C(O)—C₁-C₄-haloalkyl, C(O)—C₃-C₄-cycloalkyl, C(O)—C₃-C₄-halo¬cyclo¬alkyl;
R² is H, CN, C₁-C₃-alkyl, C₁-C₃-haloalkyl, C₂-C₃-alkynyl;
R³ is pyridine, pyrimidine, pyrazine, or pyridazine, which ring is unsubstituted or substituted with (R¹¹)ₙ and/or 1 to 3 halogen;
   n is 0, 1, 2, or 3;
W is N or C—R⁴;
with the proviso that W is not C—R⁴, if R³ is pyridine;
   R⁴ are independently from each other H, halogen, OH, CN, C₁-C₄-alkyl, C₁-C₄-haloalkyl, C₂-C₄-alkenyl, C₂-C₄-halo¬alkenyl, C₂-C₄-alkynyl, C₁-C₄-alkoxy, C₁-C₄-haloalkoxy, S(O)ₘ—C₁-C₄-alkyl, S(O)ₘ-C₁-C₄-haloalkyl, S(O)ₘ—C₃-C₄-cyclo¬alkyl, S(O)ₘ—C₁-C₄-halocyclo¬alkyl,
Q is a 5- to 10-membered heteroaryl comprising as ring members 1 to 4 heteroatoms selected from N, O and S which may be oxidized, wherein at least one ring member heteroatom is N, which heteroaryl is unsubstituted, or partially or fully substituted with groups independently selected from R⁵;
R⁵ halogen, OH, CN, SF₅, COOH, CONH₂, NO₂, or C₁-C₆-alkyl, C₃-C₆-cycloalkyl, C₃-C₆-cyclo-alkyl-C₁-C₆-alkyl, C₁-C₃-haloalkyl, C₁-C₄-alkoxy, C₁-C₃-haloalkoxy, S(O)ₘ—C₁-C₆-alkyl, S(O)ₘ—C₃-C₆-cycloalkyl, S(O)ₘ—C₁-C₃-haloalkyl, S(O)ₘ-phenyl, NR¹²R¹³, NR¹²CO—C₁-C₄-alkyl, NHCO-phenyl, CO₂—C₁-C₄-alkyl, CONR¹²R¹³, CONR¹²(C₃-C₆-cycloalkyl), C(═NO—C₁-C₄-alkyl)R¹²; phenyl and 5- to 6-membered heteroaryl, wherein aromatic rings are unsubstituted, or substituted with 1 to 2 halogen and/or CN; R⁵ groups being unsubstituted, or partially or fully substituted with R¹¹;
two R⁵ present on the same carbon atom may together form a group ═O, ═S, ═NH, ═N(C₁-C₆-alkyl), ═NO(C₁-C₆-alkyl), ═NN(H)(C₁-C₆-alkyl), or ═NN(C₁-C₆-alkyl)₂;
and the N-oxides, stereoisomers and agriculturally or veterinarily acceptable salts thereof.

The invention also provides agricultural compositions comprising at least one compound of formula I, a stereoisomer thereof and/or an agriculturally acceptable salt thereof and at least one liquid and/or solid carrier, especially at least one inert liquid and/or solid agriculturally acceptable carrier.

The invention also provides a veterinary composition comprising at least one compound of formula I, a stereoisomer thereof and/or a veterinarily acceptable salt thereof and at least one liquid and/or solid carrier, especially at least one inert veterinarily liquid and/or solid acceptable carrier.

The invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The invention also relates to plant propagation material, in particular seed, comprising at least one compound of formula I and/or an agriculturally acceptable salt thereof.

The invention further relates to a method for treating or protecting an animal from infestation or infection by parasites which comprises bringing the animal in contact with a parasiticidally effective amount of a compound of formula I or a veterinarily acceptable salt thereof. Bringing the animal in contact with the compound I, its salt or the veterinary composition of the invention means applying or administering it to the animal.

WO2017/192385, WO2019/197468, WO2019/202077, WO2019/201835, WO2019/206799, WO2020/002563, and WO2020/212235 describe structurally closely related active compounds. These compounds are mentioned to be useful for combating invertebrate pests.

Nevertheless, there remains a need for highly effective and versatile agents for combating invertebrate pests. It is therefore an object of the invention to provide compounds having a good pesticidal activity and showing a broad activity spectrum against a large number of different invertebrate pests, especially against difficult to control pests, such as insects.

It has been found that these objects can be achieved by compounds of formula I as depicted and defined below, and by their stereoisomers, salts, tautomers and N-oxides, in particular their agriculturally acceptable salts.

Compounds I can be obtained by alkylation of a compound II with a suitable alkylating agent III (e.g. alkyl halide). In formula III R¹ has the meaning as in formula I, and X is a nucleophilic leaving group, such as a halide, preferably Br or Cl. The alkylation can be effected under standard conditions known from literature.

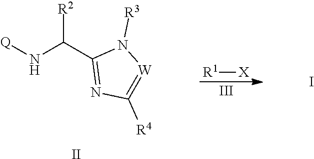

This transformation is usually carried out at temperatures of from −10° C. to +110° C., preferably from 0° C. to 25° C., in an inert solvent, in the presence of a base [cf. WO 2002100846].

The starting materials are generally reacted with one another in equimolar amounts. In terms of yield, it may be advantageous to employ an excess of III, based on II.

Compounds II can be obtained by reduction of an imine of formula IV. The reduction can be effected under standard conditions known from literature.

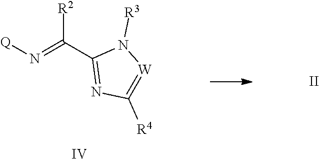

This transformation is usually carried out at temperatures of from −20° C. to 120° C., preferably from 0° C. to 60° C., in an inert solvent, in the presence of a reducing agent [cf. WO2016/201096]

Suitable reducing agents are e.g. alkali metal borohydrides such as NaBH₄, NaBH(OAc)₃, NaBH₃CN, and the like. Suitable solvents are e.g. alcohols such as methanol, ethanol, n-propanol, isopropanol, n-butanol, and tert-butanol; or aromatic hydrocarbons such as toluene, o-, m-, and p-xylene; or halogenated hydrocarbons such as methylene chloride, 1,2-dichloroethane, chloroform, and chlorobenzene; or ethers such as 1,4-dioxane, and tetrahydrofuran (THF), and it is also possible to use mixtures of the solvents mentioned. The reducing agent is usually employed in equimolar amounts. In terms of yield, it may be advantageous to employ an excess of reducing agent, based on IV.

Compounds of formula IV can be obtained by reaction of a ketone V with an amine of formula VI under conditions known from literature.

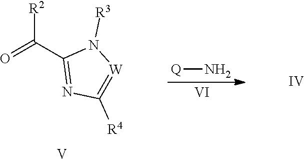

This transformation is usually carried out at temperatures of from 25° C. to 220° C., preferably from 60° C. to 150° C., in an inert solvent, in the presence of an acid “HJ” [cf. WO2016/201096].

Suitable solvents are e.g. aromatic hydrocarbons such as toluene, o-, m-, and p-xylene; or alcohols such as methanol, ethanol, n-propanol, isopropanol, n-butanol, and tert-butanol; or nitriles such as acetonitrile, and propionitrile; or ethers such as 1,4-dioxane, and THF; or halogenated hydrocarbons such as methylene chloride, chloroform, and chlorobenzene. It is also possible to use mixtures of the solvents mentioned. Suitable acids and acidic catalysts are in general organic acids, e.g. carboxylic acids such as formic acid, acetic acid, propionic acid, oxalic acid, and trifluoroacetic acid; or sulfonic acids such as methanesulfonic acid, toluenesulfonic acid, benzenesulfonic acid, camphor sulfonic acid, and trifluoromethanesulfonic acid; or Lewis acids, such as e.g. titanium(IV) ethoxide, titanium(IV) isopropoxide, titanium(IV) chloride, boron trifluoride, and zinc(II) chloride; or inorganic acids such as sulfuric acid, and hydrochloric acid. The acids are generally employed in catalytic amounts; however, they can also be used in equimolar amounts, in excess or, if appropriate, as solvent. It is furthermore understood by a person skilled in the art, that water, which is formed in the course of the reaction, can be continuously removed by means of a Dean-Stark trap or a drying agent such as e.g. MgSO₄ or molecular sieves. The starting materials are generally reacted with one another in equimolar amounts. In terms of yield, it may be advantageous to employ an excess of amine VI, based on V.

Compounds of formula V can be obtained by reaction of an imidazole or triazole derivative VI with a base, followed by an acylating agent R²—C(═O)Y of formula VII, wherein Y is a suitable leaving group such as e.g. halogen, N(CH₃)₂, NCH₃(OCH₃), alkoxy, or the like, under conditions known from literature.

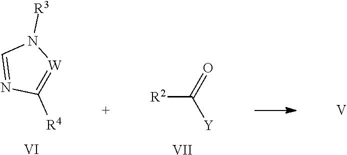

This transformation is usually carried out at temperatures of from −100° C. to 80° C., preferably from −78° C. to 40° C., in an inert solvent, in the presence of a base [cf. WO2014/100163, Chemical Communications, 2016, vol. 52, p. 10183-10186].

Suitable solvents are aliphatic hydrocarbons such as pentane, hexane, cyclohexane, and petrol ether; or aromatic hydrocarbons such as toluene, o-, m-, and p-xylene; or ethers such as diethyl ether, diisopropyl ether, tert-butylmethyl ether (MTBE), and THF; or halogenated hydrocarbons such as methylene chloride, chloroform, and chlorobenzene; or nitriles such as acetonitrile, and propionitrile. It is also possible to use mixtures of the solvents mentioned. Suitable bases are, in general, organic bases, e.g. alkali metal alkyl bases such as n-BuLi; or alkali metal and alkaline earth metal amides such as e.g. lithium diisopropylamide, lithium bis(trimethylsilyl)amide, lithium tetramethylpiperidide, and iPr₂NMgCl·LiCl; or tertiary amines, such as trimethylamine, triethylamine, diisopropylethylamine and N-methylpiperidine, pyridine, substituted pyridines, such as collidine, lutidine, and 4-dimethylaminopyridine, and also bicyclic amines. The bases are generally employed in equimolar amounts; however, they can also be used in excess or, if appropriate, as solvent. The starting materials are generally reacted with one another in equimolar amounts. In terms of yield, it may be advantageous to employ an excess of VII, based on VI. Compounds of formula VI are commercially available, or can be prepared as described in literature, or analogously to the synthesis examples.

Alternatively, ketones V can be obtained from compounds VIII, wherein X^{#} is a leaving group such as a halogen, by reacting it with a sulfoxide such as dimethyl sulfoxide (DMSO); or a suitable N-oxide, such as e.g. trimethylamine N-oxide or pyridine N-oxide, in a Kornblum oxidation under conditions known from literature.

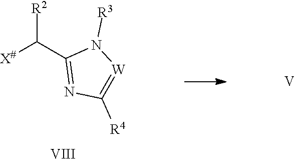

This transformation is usually carried out at temperatures of from 25° C. to 220° C., preferably from 25° C. to 120° C., in an inert solvent, in the presence of a base [cf. JP2019/34892, WO2006/110804].

Suitable solvents are DMSO, dimethyl formamide (DMF), and dimethylacetamide (DMA); or nitriles such as acetonitrile, and propionitrile; or ethers such as 1,4-dioxane, and THF; or halogenated hydrocarbons such as methylene chloride, and chloroform; or water. It is also possible to use mixtures of the solvents mentioned. Suitable bases are, in general, inorganic compounds, such as e.g. alkali metal and alkaline earth metal carbonates, such as Li₂CO₃, K₂CO₃, Cs₂CO₃, and CaCO₃; or alkali metal bicarbonates, such as e.g. NaHCO₃; or organic bases, e.g. tertiary amines, such as trimethylamine, triethylamine, diisopropylethylamine and N-methylpiperidine, pyridine, substituted pyridines, such as collidine, lutidine, and also bicyclic amines. The sulfoxide or N-oxide is generally employed in excess; however, if appropriate, it can also be used as solvent.

Compounds of formula VIII can be prepared as described in the literature [cf. WO2017/192385, WO2019/197468, WO2019/202077, WO2019/201835, WO2019/206799, WO 2020/002563].

Alternatively compounds I can be obtained directly by reduction of intermediately formed iminium compounds IX, which in turn are accessible from ketones V by reaction with an amine Q-NHR¹ of formula X. The reaction conditions are analogous as described above for the transformations of compounds V into compounds of formula IV, and then II, respectively. J⁻ is the anion of the acid HJ used.

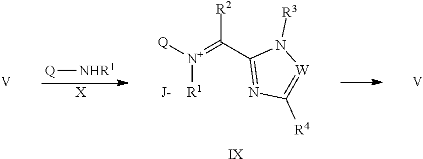

Moreover, compounds I can also be obtained via nucleophilic aromatic substitution or Buchwald-Hartwig reaction from an amine of formula XI with a halide XII, wherein X* is a leaving group such as a halogen or a triflate.

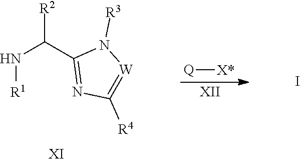

Nucleophilic aromatic substitution reactions are usually carried out at temperatures from −20° C. to 180° C., preferably from 25° C. to 100° C., in an inert solvent, and in the presence of a base [cf. WO 2010/100189].

Suitable solvents are e.g. DMSO, DMF, and DMA; or nitriles such as acetonitrile, and propionitrile; or ethers such as 1,4-dioxane, and THF; or aromatic hydrocarbons such as toluene, o-, m-, and p-xylene; or alcohols such as methanol, ethanol, n-propanol, isopropanol, n-butanol, and tert-butanol; or water. Suitable bases are, in general, inorganic compounds, such as alkali metal and alkaline earth metal carbonates, such as Li₂CO₃, K₂CO₃, Cs₂CO₃, and CaCO₃; or alkali metal and alkaline earth metal hydrides, such as LiH, NaH, KH, and CaH₂; or organic bases, e.g. tertiary amines, such as trimethylamine, triethylamine, diisopropylethylamine and N-methylpiperidine, pyridine, substituted pyridines, such as collidine, lutidine and 4-dimethylaminopyridine, and also bicyclic amines; or alkali metal alcoholates such as potassium tert-butoxide. The bases are generally employed in equimolar amounts; however, they can also be used in excess or, if appropriate, as solvent. The starting materials are generally reacted with one another in equimolar amounts. In terms of yield, it may be advantageous to employ an excess of XII, based on XI.

Buchwald-Hartwig reactions are usually carried out at temperatures from 25° C. to 220° C., preferably from 50° C. to 150° C., in an inert solvent, and in the presence of a palladium catalyst and a base [cf. WO2016/168059].

Suitable solvents are e.g. aromatic hydrocarbons such as toluene, o-, m-, and p-xylene; or ethers such as 1,4-dioxane, and THF; or nitriles such as acetonitrile, and propionitrile; or polar aprotic solvents such as DMSO, DMF, DMA, and N-methyl-2-pyrrolidon (NMP).

Suitable palladium catalysts are e.g. Pd(OAc)₂/PPh₃, Pd(OAc)₂/2,2′-bis(diphenylphosphino)-1,1′-binaphthyl (BINAP), Pd(OAc)₂/4,5-bis(diphenylphosphino)-9,9-dimethylxanthene (XantPhos), dichloro[9,9-dimethyl-4,5-bis(diphenylphosphino)xanthene]palladium(II), [1,1′-bis(diphenylphosphino)ferrocene]dichloropalladium(II), (1,3-bis(diphenylphosphino)propane)palladium(II) chloride, trans-bis(acetato)bis[o-(di-o-tolylphosphino)benzyl]dipalladium(II), and the like.

Suitable bases are, in general, inorganic compounds, such as alkali metal and alkaline earth metal carbonates, such as Li₂CO₃, K₂CO₃, Cs₂CO₃, and CaCO₃; or alkali metal phosphates such as K₃PO₄; or organic bases, e.g. tertiary amines, such as triethylamine, diisopropylethylamine, N-methylpiperidine, N-methyl-N,N-dicyclohexylamine, and 1,4-diaza-bicyclo[2.2.2]octane; or amidines such as 1,8-diazabicyclo[5.4.0]undec-7-en; or alkali metal alcoholates such as sodium tert-butoxide; or alkali metal amides such as lithium bis(trimethylsilyl)amide (LiHMDS). The bases are generally employed in equimolar amounts; however, they can also be used in excess or, if appropriate, as solvent.

The starting materials are generally reacted with one another in equimolar amounts. In terms of yield, it may be advantageous to employ an excess of XI, based on XII.

Amines of formula XI can be prepared as described [cf. WO2017/192385, WO2019/197468, WO2019/202077, WO2019/201835, WO2019/206799, and WO 2020/002563]. Halides of formula XII are commercially available, can be prepared as described in literature, or in analogy to the synthesis examples.

The reaction mixtures are worked up in a customary manner, for example by mixing with water, separating the phases and, if appropriate, chromatographic purification of the crude products. Some of the intermediates and end products are obtained in the form of colourless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

However, if the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the pest to be controlled.

The organic moieties groups mentioned in the above definitions of the variables are—like the term halogen—collective terms for individual listings of the individual group members. The prefix Cₙ-Cₘ indicates in each case the possible number of carbon atoms in the group.

The term “partially or fully substituted” by a radical means that in general the group is substituted with same or different radicals.

The term “halogen” denotes in each case fluorine, bromine, chlorine, or iodine, in particular fluorine, chlorine, or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl (Me), ethyl (Et), n-propyl (n-Pr), iso-propyl, n-butyl, 2-butyl, iso-butyl, tert-butyl, n-pentyl, 1-methylbutyl, 2-methylbutyl, 3-methylbutyl, 2,2-dimethyl propyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂-OC₂H₅, 2-(methoxy)ethyl, and 2-(ethoxy)ethyl.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms. Preferred haloalkoxy moieties include C₁-C₄-haloalkoxy, in particular C₁-C₂-fluoroalkoxy, such as fluoromethoxy, difluoromethoxy, trifluoromethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2dichloro-2-fluorethoxy, 2,2,2-trichloroethoxy, pentafluoroethoxy and the like.

The term “alkylthio “(alkylsulfanyl: alkyl-S-)” as used herein refers to a straight-chain or branched saturated alkyl group having 1 to 10 carbon atoms, preferably 1 to 4 carbon atoms (═C₁-C₄-alkylthio), more preferably 1 to 3 carbon atoms, which is attached via a sulfur atom.

The term “haloalkylthio” as used herein refers to an alkylthio group as mentioned above wherein the hydrogen atoms are partially or fully substituted by fluorine, chlorine, bromine and/or iodine.

The term “alkylsulfinyl” (alkylsulfoxyl: C₁-C₆-alkyl-S(═O)—), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “haloalkylsulfinyl” as used herein refers to an alkylsulfinyl group as mentioned above wherein the hydrogen atoms are partially or fully substituted by fluorine, chlorine, bromine and/or iodine.

The term “alkylsulfonyl” (alkyl-S(═O)₂—) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “haloalkylsulfonyl” as used herein refers to an alkylsulfonyl group as mentioned above wherein the hydrogen atoms are partially or fully substituted by fluorine, chlorine, bromine and/or iodine.

The term “alkylcarbonyl” refers to an alkyl group as defined above, which is bonded via the carbon atom of a carbonyl group (C═O) to the remainder of the molecule.

The term “haloalkylcarbonyl” refers to an alkylcarbonyl group as mentioned above, wherein the hydrogen atoms are partially or fully substituted by fluorine, chlorine, bromine and/or iodine.

The term “alkoxycarbonyl” refers to an alkylcarbonyl group as defined above, which is bonded via an oxygen atom to the remainder of the molecule.

The term “haloalkoxycarbonyl” refers to an alkoxycarbonyl group as mentioned above, wherein the hydrogen atoms are partially or fully substituted by fluorine, chlorine, bromine and/or iodine.

The term “alkenyl” as used herein denotes in each case a singly unsaturated hydrocarbon radical having usually 2 to 10, frequently 2 to 6, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radical having usually 2 to 10, frequently 2 to 6, preferably 2 to 4 carbon atoms, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “cycloalkyl” as used herein and in the cycloalkyl moieties of cycloalkoxy and cycloalkylthio denotes in each case a monocyclic cycloaliphatic radical having usually from 3 to 10 or from 3 to 6 carbon atoms, such as cyclopropyl (c-C₃H₅), cyclobutyl, cyclopentyl, cyclohexyl, cycloheptyl, cyclooctyl, cyclononyl and cyclodecyl or cyclopropyl, cyclobutyl, cyclopentyl and cyclohexyl.

The term “halocycloalkyl” as used herein and in the halocycloalkyl moieties of halocycloalkoxy and halocycloalkylthio denotes in each case a monocyclic cycloaliphatic radical having usually from 3 to 10 C atoms or 3 to 6 C atoms, wherein at least one, e.g. 1, 2, 3, 4 or 5 of the hydrogen atoms, are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “halocycloalkenyl” as used herein and in the halocycloalkenyl moieties of halocycloalkenyloxy and halocycloalkenylthio denotes in each case a monocyclic singly unsaturated non-aromatic radical having usually from 3 to 10, e.g. 3 or 4 or from 5 to 10 carbon atoms, preferably from 3- to 8 carbon atoms, wherein at least one, e.g. 1, 2, 3, 4 or 5 of the hydrogen atoms, are replaced by halogen, in particular by fluorine or chlorine. Examples are 3,3-difluorocyclopropen-1-yl and 3,3-dichlorocyclopropen-1-yl.

The term “cycloalkenylalkyl” refers to a cycloalkenyl group as defined above which is bonded via an alkyl group, such as a C₁-C₅-alkyl group or a C₁-C₄-alkyl group, in particular a methyl group (═cycloalkenylmethyl), to the remainder of the molecule.

The term “carbocycle” or “carbocyclyl” includes in general a 3- to 12-membered, preferably a 3- to 8-membered or a 5- to 8-membered, more preferably a 5- or 6-membered mono-cyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 3- to 6-membered, in particular 6-membered monocyclic heterocyclic non-aromatic radicals. The heterocyclic non-aromatic radicals usually comprise 1, 2, 3, 4 or 5, preferably 1, 2 or 3 heteroatoms selected from N, O and S as ring members, wherein S-atoms as ring members may be present as S, SO or SO₂. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S. oxothiopyranyl, S-dioxothiopyranyl, di hydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-onyl and the like.

The term “hetaryl” includes monocyclic 5- or 6-membered heteroaromatic radicals comprising as ring members 1, 2, 3 or 4 heteroatoms selected from N, O and S. Examples of 5- or 6-membered heteroaromatic radicals include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl. The term “hetaryl” also includes bicyclic 8 to 10-membered heteroaromatic radicals comprising as ring members 1, 2 or 3 heteroatoms selected from N, O and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzothienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like. These fused hetaryl radicals may be bonded to the remainder of the molecule via any ring atom of 5- or 6-membered heteroaromatic ring or via a carbon atom of the fused phenyl moiety.

The terms “heterocyclylalkyl” and “hetarylalkyl” refer to heterocyclyl or hetaryl, respectively, as defined above which are bonded via a C₁-C₅-alkyl group or a C₁-C₄-alkyl group, in particular a methyl group (=heterocyclylmethyl or hetarylmethyl, respectively), to the remainder of the molecule.

The term “arylalkyl” and “phenylalkyl” refer to aryl as defined above and phenyl, respectively, which are bonded via C₁-C₅-alkyl group or a C₁-C₄-alkyl group, in particular a methyl group (═arylmethyl or phenylmethyl), to the remainder of the molecule, examples including benzyl, 1-phenylethyl, 2-phenylethyl, 2-phenoxyethyl etc.

The terms “alkylene”, “cycloalkylene”, “heterocycloalkylene”, “alkenylene”, “cycloalkenylene”, “heterocycloalkenylene” and “alkynylene” refer to alkyl, cycloalkyl, heterocycloalkyl, alkenyl, cycloalkenyl, heterocycloalkenyl and alkynyl as defined above, respectively, which are bonded to the remainder of the molecule, via two atoms, preferably via two carbon atoms, of the respective group, so that they represent a linker between two moieties of the molecule.

In a particular embodiment, the variables of the compounds of the formula I have the following meanings, these meanings, both on their own and in combination with one another, being particular embodiments of the compounds of the formula I.

Embodiments and preferred compounds of the invention for use in pesticidal methods and for insecticidal application purposes are outlined in the following paragraphs.

With respect to the variables, the particularly preferred embodiments of the intermediates correspond to those of the compounds of the formula I.

In a preferred embodiment, the compounds I are present in form of a mixture of compounds I.A and I.B, wherein compound I.A with S-configuration of the carbon atom neighboring the nitrogen is present in an amount of more than 50% by weight, in particular of at least 70% by weight, more particularly of at least 85% by weight, specifically of at least 90% by weight, based on the total weight of compounds I.A and I.B.

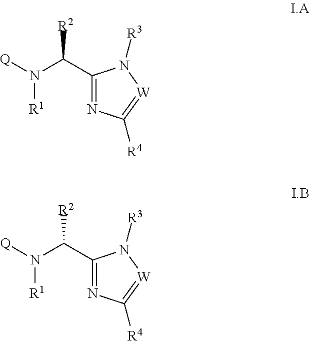

In one particularly preferred embodiment of the invention, the method comprises the step of contacting the plant, parts of it, its propagation material, the pests, their food supply, habitat or breeding grounds a pesticidally effective amount of a compound of formula I.A.

Preferably R¹ is H, OH, C₁-C₄-alkoxy-C₁-C₂-alkyl, C(═O)R^{11a} with R^{11a} being c-C₃H₅CH₂, C₁-C₄-alkoxy, C₁-C₄-alkyl, C₁-C₄-haloalkyl, C₃-C₆-cycloalkyl, C₃-C₄-cycloalkyl-C₁-C₂-alkyl, and OR¹⁴.

Particularly R¹ is H, C₁-C₄-alkoxy-C₁-C₂-alkyl, or C(═O)R^{11a} with R^{11a} being C₁-C₄-alkyl, C₁-C₄-haloalkyl, C₃-C₆-cycloalkyl, C₃-C₄-cycloalkyl-C₁-C₂-alkyl, or OR¹⁴. Compounds with R¹ being H are most preferred.

In preferred embodiment W is N. Such compounds correspond to formula I.N.

In another embodiment W is CR⁴. Such compounds correspond to formula I.C.

In formulae I.N and I.C, resp., the variables are as defined and preferred for formula I.

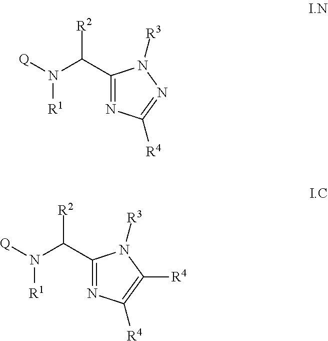

R² is preferably H, CH₃, C≡CH, or CN, particularly CH₃.

Preferably R³ is pyrimidine-2 which is substituted with (R¹¹)ₙ, such compounds correspond to formula I.1

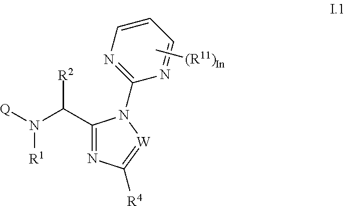

Preferably in such compounds n is 0; if n is not 0, a group R¹¹ stands preferably in meta and/or more preferred in para position.

In another embodiment R³ is pyridine-2, which is substituted with (R¹¹)ₙ, such compounds correspond to formula I.2

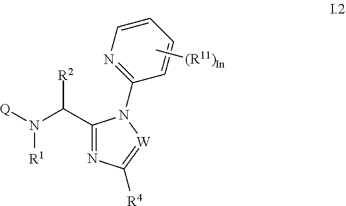

Preferably in such compounds n is 0; if n is not 0, a group R¹¹ stands preferably in meta and/or more preferred in para position.

R¹¹ in general, and particularly in compounds I.1 and I.2, are preferably selected from halogen, CN, halomethoxy, and halomethyl, such as CN, CI, F, Br, CF₃, OCF₃, and OCHF₂.

R⁴ is preferably H, halogen, or CH₃, particularly H.

Q is preferably a N containing 5- to 10-membered heteroaryl comprising as ring members at least one N atom and 1, to 3 heteroatoms selected from N, O and S which may be oxidized, which heteroaryl ring is unsubstituted, or partially or fully substituted with groups independently selected from R⁵.

Q is more preferably a N containing 5- to 10-membered heteroaryl comprising as ring members at least one N atom and 1, or 2 heteroatoms selected from N, O and S which may be oxidized of structure QN.

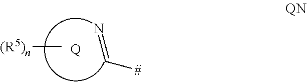

In one embodiment Q is a bicyclic 9-membered hetaryl with 2 or 3 heteroatoms as ring members selected from N, O, and S. Such compounds correspond preferably to formula I.X

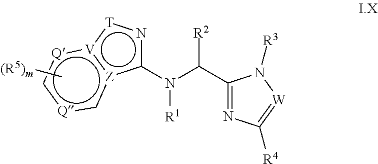

wherein

T is CH, CR⁵, N, O, or S which may be oxidized;

V is C or N;

Z is C or N;

Q′ is CH, CR⁵, or N; and

Q″ is CH, CR⁵, or N;

with the proviso that 2 or 3 of T, V, Z, Q′, and Q″ are a heteroatom.

Preferred hetaryls in formula I.X are benzoxazole, imidazole, 1,2-benzothiazole, isoxazolo[5,4-b]pyridine, 1lambda5,7,8-triazabicyclo[4.3.0]nona-1(6),2,4,8-tetraene.

In particularly preferred compounds of formula I.X R³ is 2-pyrimidine, which is unsubstituted or substituted in para position with R^{11A}, W is N, and V is C. R^{11A} is H or R¹¹. Such compounds correspond to formula I.X*

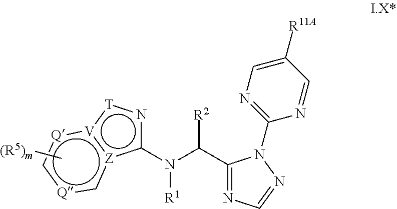

In another embodiment Q is a 5-membered hetaryl containing at least one N as ring member, which hetaryl is unsubstituted or partially or fully substituted with groups independently selected from R⁵. Such 5-membered hetaryl is preferably pyrazole, isoxazole, isothiazole, imidazole, triazole; preferably 5-pyrazole, 3-isoxazole, 3-isothiazole, 5-isoxazole, 5-isothiazole, 4-imidazole, or 1,4-dihydro-1,2,4-triazol-5-one.

In another embodiment Q is a thiophene.

In another embodiment Q is a 6-membered hetaryl containing at least one N as ring member, which hetaryl is unsubstituted or partially or fully substituted with groups independently selected from R⁵. Such 5-membered hetaryl is preferably pyridine, pyrimidine, or pyridazine, preferably 2-pyridine, 2-pyrimidin, or 3-pyridazine.

Particularly preferred embodiments are Q selected from Q1 to Q18

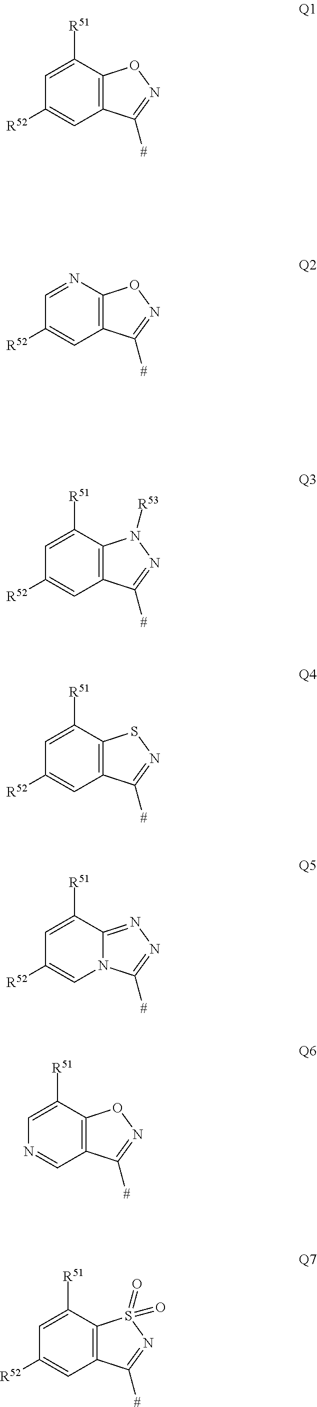

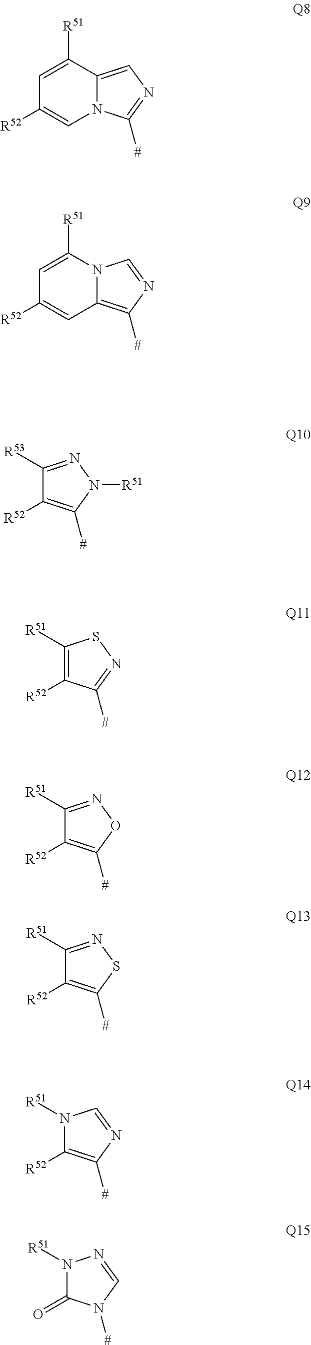

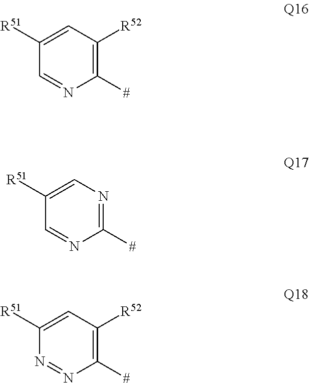

wherein R⁵¹, R⁵², R⁵³ are independently from each other H or R⁵; and

# is the bond to the remainder of the molecule

Other preferred embodiments are Q selected from Q19 and Q20, wherein R⁵¹ and R⁵² are independently from each other H or R⁵; and # is the bond to the remainder of the molecule

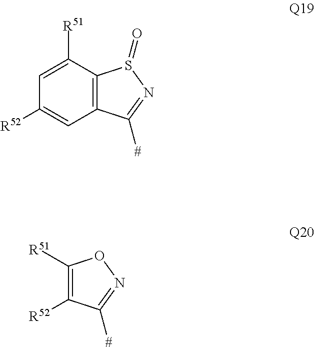

R⁵ is preferably halogen, CN, C₁-C₄-alkyl, C₁-C₂-haloalkyl, C₁-C₂-haloalkoxy, S(O)ₘ—C₁-C₄-alkyl, S(O)ₘ—C₁-C₄-haloalkyl, S(O)ₘ—C₃-C₄-cycloalkyl, S(O)ₘ-C₃-C₄-halocycloalkyl, C₃-C₄-cycloalkyl, C₃-C₄-cycloalkyl substituted with CN, halogen, or C₁-C₂-haloalkyl; more preferably R⁵ is Cl, S(O)CH₃, S(O)₂CH₃, S(O)CF₃, S(O)₂CF₃, c-C₃H₅, c-C₃H₄-CF₃, c-C₃H₄—CN, or halomethyl such as CF₃.

In a particular embodiment, the variables of the compounds of the formula I have the following meanings, these meanings, both on their own and in combination with one another, being particular embodiments of the compounds of the formula I:

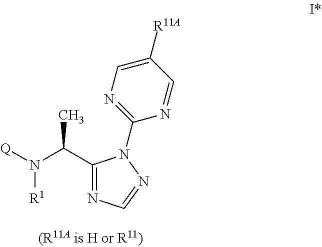

Table 1
Compounds of formula I* in which R¹ is H, R^{11A} is H, and the combination of Q, R⁵¹, R⁵², and R⁵³ for a compound corresponds in each case to one row of Table A

Table 2
Compounds of formula I* in which R¹ is c-C₃H₅CH₂, R^{11A} is H, and the combination of R^{11A}, Q R⁵¹, R⁵², and R⁵³ for a compound corresponds in each case to one row of Table A

Table 3
Compounds of formula I* in which R¹ is C₂H₅OCH₂, R^{11A} is H, and the combination of R^{11A}, Q R⁵¹, R⁵², and R⁵³ for a compound corresponds in each case to one row of Table A

Table 4
Compounds of formula I* in which R¹ is C(═O)OCH₂-c-C₃H₅, R^{11A} is H, and the combination of R^{11A}, Q, R⁵¹, R⁵², and R⁵³ for a compound corresponds in each case to one row of Table A

Table 5
Compounds of formula I* in which R¹ is C(═O)CH₂-c-C₃H₅, R^{11A} is H, and the combination of R^{11A}, Q, R⁵¹, R⁵², and R⁵³ for a compound corresponds in each case to one row of Table A

Table 6
Compounds of formula I* in which R¹ is OH, R^{11A} is H, and the combination of R^{11A}, Q, R⁵¹, R⁵², and R⁵³ for a compound corresponds in each case to one row of Table A

Table 7
Compounds of formula I* in which R¹ is H, R^{11A} is Br, and the combination of Q, R⁵¹, R⁵², and R⁵³ for a compound corresponds in each case to one row of Table A

Table 8
Compounds of formula I* in which R¹ is c-C₃H₅CH₂, R^{11A} is Br, and the combination of R^{11A}, Q R⁵¹, R⁵², and R⁵³ for a compound corresponds in each case to one row of Table A

Table 9
Compounds of formula I* in which R¹ is C₂H₅OCH₂, R^{11A} is Br, and the combination of R^{11A}, Q R⁵¹, R⁵², and R⁵³ for a compound corresponds in each case to one row of Table A

Table 10
Compounds of formula I* in which R¹ is C(═O)OCH₂-c-C₃H₅, R^{11A} is Br, and the combination of R^{11A}, Q, R⁵¹, R⁵², and R⁵³ for a compound corresponds in each case to one row of Table A

Table 11
Compounds of formula I* in which R¹ is C(═O)CH₂-c-C₃H₅, R^{11A} is Br, and the combination of R^{11A}, Q, R⁵¹, R⁵², and R⁵³ for a compound corresponds in each case to one row of Table A

Table 12
Compounds of formula I* in which R¹ is OH, R^{11A} is Br, and the combination of R^{11A}, Q, R⁵¹, R⁵², and R⁵³ for a compound corresponds in each case to one row of Table A

Table 13
Compounds of formula I* in which R¹ is H, R^{11A} is Cl, and the combination of Q, R⁵¹, R⁵², and R⁵³ for a compound corresponds in each case to one row of Table A

Table 14
Compounds of formula I* in which R¹ is c-C₃H₅CH₂, R^{11A} is Cl, and the combination of R^{11A}, Q R⁵¹, R⁵², and R⁵³ for a compound corresponds in each case to one row of Table A

Table 15
Compounds of formula I* in which R¹ is C₂H₅OCH₂, R^{11A} is Cl, and the combination of R^{11A}, Q R⁵¹, R⁵², and R⁵³ for a compound corresponds in each case to one row of Table A

Table 16
Compounds of formula I* in which R¹ is C(═O)OCH₂-c-C₃H₅, R^{11A} is Cl, and the combination of Q, R⁵¹, R⁵², and R⁵³ for a compound corresponds in each case to one row of Table A

Table 17
Compounds of formula I* in which R¹ is C(═O)CH₂-c-C₃H₅, R^{11A} is Cl, and the combination of Q, R⁵¹, R⁵², and R⁵³ for a compound corresponds in each case to one row of Table A

Table 18
Compounds of formula I* in which R¹ is OH, R^{11A} is Cl, and the combination of R^{11A}, Q, R⁵¹, R⁵², and R⁵³ for a compound corresponds in each case to one row of Table A

Table 19
Compounds of formula I* in which R¹ is H, R^{11A} is F, and the combination of Q, R⁵¹, R⁵², and R⁵³ for a compound corresponds in each case to one row of Table A

Table 20
Compounds of formula I* in which R¹ is c-C₃H₅CH₂, R^{11A} is F, and the combination of R^{11A}, Q R⁵¹, R⁵², and R⁵³ for a compound corresponds in each case to one row of Table A

Table 21
Compounds of formula I* in which R¹ is C₂H₅OCH₂, R^{11A} is F, and the combination of R^{11A}, Q R⁵¹, R⁵², and R⁵³ for a compound corresponds in each case to one row of Table A

Table 22
Compounds of formula I* in which R¹ is C(═O)OCH₂-c-C₃H₅, R^{11A} is F, and the combination of R^{11A}, Q, R⁵¹, R⁵², and R⁵³ for a compound corresponds in each case to one row of Table A

Table 23
Compounds of formula I* in which R¹ is C(═O)CH₂-c-C₃H₅, R^{11A} is F, and the combination of R^{11A}, Q, R⁵¹, R⁵², and R⁵³ for a compound corresponds in each case to one row of Table A

Table 24
Compounds of formula I* in which R¹ is OH, R^{11A} is F, and the combination of R^{11A}, Q, R⁵¹, R⁵², and R⁵³ for a compound corresponds in each case to one row of Table A

Table 25
Compounds of formula I* in which R¹ is H, R^{11A} is CF₃, and the combination of Q, R⁵¹, R⁵², and R⁵³ for a compound corresponds in each case to one row of Table A

Table 26
Compounds of formula I* in which R¹ is c-C₃H₅CH₂, R^{11A} is CF₃, and the combination of R^{11A}, Q R⁵¹, R⁵², and R⁵³ for a compound corresponds in each case to one row of Table A

Table 27
Compounds of formula I* in which R¹ is C₂H₅OCH₂, R^{11A} is CF₃, and the combination of R^{11A}, Q R⁵¹, R⁵², and R⁵³ for a compound corresponds in each case to one row of Table A

Table 28
Compounds of formula I* in which R¹ is C(═O)OCH₂-c-C₃H₅, R^{11A} is CF₃, and the combination of R^{11A}, Q, R⁵¹, R⁵², and R⁵³ for a compound corresponds in each case to one row of Table A

Table 29
Compounds of formula I* in which R¹ is C(═O)CH₂-c-C₃H₅, R^{11A} is CF₃, and the combination of R^{11A}, Q, R⁵¹, R⁵², and R⁵³ for a compound corresponds in each case to one row of Table A

Table 30
Compounds of formula I* in which R¹ is OH, R^{11A} is CF₃, and the combination of R^{11A}, Q, R⁵¹, R⁵², and R⁵³ for a compound corresponds in each case to one row of Table A

Table 31
Compounds of formula I* in which R¹ is H, R^{11A} is CN, and the combination of Q, R⁵¹, R⁵², and R⁵³ for a compound corresponds in each case to one row of Table A

Table 32
Compounds of formula I* in which R¹ c-C₃H₅CH₂, R^{11A} is CN, and the combination of R^{11A}, Q R⁵¹, R⁵², and R⁵³ for a compound corresponds in each case to one row of Table A

Table 33
Compounds of formula I* in which R¹ is C₂H₅OCH₂, R^{11A} is CN, and the combination of R^{11A}, Q R⁵¹, R⁵², and R⁵³ for a compound corresponds in each case to one row of Table A

Table 34
Compounds of formula I* in which R¹ is C(═O)OCH₂-c-C₃H₅, R^{11A} is CN, and the combination of R^{11A}, Q, R⁵¹, R⁵², and R⁵³ for a compound corresponds in each case to one row of Table A

Table 35
Compounds of formula I* in which R¹ is C(═O)CH₂-c-C₃H₅, R^{11A} is CN, and the combination of R^{11A}, Q, R⁵¹, R⁵², and R⁵³ for a compound corresponds in each case to one row of Table A

Table 36
Compounds of formula I* in which R¹ is OH, R^{11A} is CN, and the combination of R^{11A}, Q, R⁵¹, R⁵², and R⁵³ for a compound corresponds in each case to one row of Table A

TABLE A

| No. | Q | R⁵¹ | R⁵² | R⁵³ |
|---|---|---|---|---|
| A-1 | Q1 | Cl | Cl | — |
| A-2 | Q1 | CF₃ | CF₃ | — |
| A-3 | Q1 | Cl | CF₃ | — |
| A-4 | Q1 | CF₃ | Cl | — |
| A-5 | Q2 | Cl | — | — |
| A-6 | Q2 | CF₃ | — | — |
| A-7 | Q3 | CF₃ | H | CH₃ |
| A-8 | Q3 | H | CF₃ | CH₃ |
| A-9 | Q3 | CF₃ | Cl | CH₃ |
| A-10 | Q3 | Cl | CF₃ | CH₃ |
| A-11 | Q3 | Cl | Cl | CH₃ |
| A-12 | Q3 | CF₃ | CF₃ | CH₃ |
| A-13 | Q4 | Cl | Cl | — |
| A-14 | Q4 | CF₃ | CF₃ | — |
| A-15 | Q4 | Cl | CF₃ | — |
| A-16 | Q4 | CF₃ | Cl | — |
| A-17 | Q5 | Cl | Cl | — |
| A-18 | Q5 | CF₃ | CF₃ | — |
| A-19 | Q5 | Cl | CF₃ | — |
| A-20 | Q5 | CF₃ | Cl | — |
| A-21 | Q6 | Cl | — | — |
| A-22 | Q6 | CF₃ | — | — |
| A-23 | Q7 | Cl | Cl | — |
| A-24 | Q7 | CF₃ | CF₃ | — |
| A-25 | Q7 | Cl | CF₃ | — |
| A-26 | Q7 | CF₃ | Cl | — |
| A-27 | Q8 | Cl | Cl | — |
| A-28 | Q8 | CF₃ | CF₃ | — |
| A-29 | Q8 | Cl | CF₃ | — |
| A-30 | Q8 | CF₃ | Cl | — |
| A-31 | Q9 | Cl | Cl | — |
| A-32 | Q9 | CF₃ | CF₃ | — |
| A-33 | Q9 | Cl | CF₃ | — |
| A-34 | Q9 | CF₃ | Cl | — |
| A-35 | Q10 | CH₃ | CF₃ | C₂F₅ |
| A-36 | Q10 | CH₃ | C₂F₅ | CF₃ |
| A-37 | Q10 | CH₃ | Cl | C₂F₅ |
| A-38 | Q10 | CH₃ | Cl | CF₃ |
| A-39 | Q11 | H | H | — |
| A-40 | Q11 | Cl | H | — |
| A-41 | Q11 | CF₃ | H | — |
| A-42 | Q11 | CH₃ | H | — |
| A-43 | Q11 | Cl | Cl | — |
| A-44 | Q11 | CF₃ | Cl | — |
| A-45 | Q11 | CH₃ | Cl | — |
| A-46 | Q11 | Cl | CF₃ | — |
| A-47 | Q11 | CF₃ | CF₃ | — |
| A-48 | Q11 | CH₃ | CF₃ | — |
| A-49 | Q11 | Cl | CH₃ | — |
| A-50 | Q11 | CF₃ | CH₃ | — |
| A-51 | Q11 | CH₃ | CH₃ | — |
| A-52 | Q11 | H | Cl | — |
| A-53 | Q11 | H | CF₃ | — |
| A-54 | Q11 | H | CH₃ | — |
| A-55 | Q12 | H | H | — |
| A-56 | Q12 | Cl | H | — |
| A-57 | Q12 | CF₃ | H | — |
| A-58 | Q12 | CH₃ | H | — |
| A-59 | Q12 | Cl | Cl | — |
| A-60 | Q12 | CF₃ | Cl | — |
| A-61 | Q12 | CH₃ | Cl | — |
| A-62 | Q12 | Cl | CF₃ | — |
| A-63 | Q12 | CF₃ | CF₃ | — |
| A-64 | Q12 | CH₃ | CF₃ | — |
| A-65 | Q12 | Cl | CH₃ | — |
| A-66 | Q12 | CF₃ | CH₃ | — |
| A-67 | Q12 | CH₃ | CH₃ | — |
| A-68 | Q12 | H | Cl | — |
| A-69 | Q12 | H | CF₃ | — |
| A-70 | Q12 | H | CH₃ | — |
| A-71 | Q13 | H | H | — |
| A-72 | Q13 | Cl | H | — |
| A-73 | Q13 | CF₃ | H | — |
| A-74 | Q13 | CH₃ | H | — |
| A-75 | Q13 | Cl | Cl | — |
| A-76 | Q13 | CF₃ | Cl | — |
| A-77 | Q13 | CH₃ | Cl | — |
| A-78 | Q13 | Cl | CF₃ | — |
| A-79 | Q13 | CF₃ | CF₃ | — |
| A-80 | Q13 | CH₃ | CF₃ | — |
| A-81 | Q13 | Cl | CH₃ | — |
| A-82 | Q13 | CF₃ | CH₃ | — |
| A-83 | Q13 | CH₃ | CH₃ | — |
| A-84 | Q13 | H | Cl | — |
| A-85 | Q13 | H | CF₃ | — |
| A-86 | Q13 | H | CH₃ | — |
| A-87 | Q14 | CH₃ | Cl | — |
| A-88 | Q14 | CH₃ | CF₃ | — |
| A-89 | Q14 | CH₃ | CH₃ | — |
| A-90 | Q14 | CH₃ | H | — |
| A-91 | Q15 | CH₃ | — | — |
| A-92 | Q16 | Cl | H | — |
| A-93 | Q16 | Cl | Cl | — |
| A-94 | Q16 | H | Cl | — |
| A-95 | Q16 | CF₃ | H | — |
| A-96 | Q16 | CF₃ | CF₃ | — |
| A-97 | Q16 | H | CF₃ | — |
| A-98 | Q16 | CF₃ | Cl | — |
| A-99 | Q16 | Cl | CF₃ | — |
| A-100 | Q17 | Cl | — | — |
| A-101 | Q17 | CF₃ | — | — |
| A-102 | Q18 | Cl | H | — |
| A-103 | Q18 | Cl | Cl | — |
| A-104 | Q18 | H | Cl | — |
| A-105 | Q18 | CF₃ | H | — |
| A-106 | Q18 | CF₃ | CF₃ | — |
| A-107 | Q18 | H | CF₃ | — |
| A-108 | Q18 | CF₃ | Cl | — |
| A-109 | Q18 | Cl | CF₃ | — |

The term “compound(s) of the invention” refers to compound(s) of formula I, or “compound(s) I”, and includes their salts, tautomers, stereoisomers, and N-oxides.

The invention also relates to agrochemical compositions comprising an auxiliary and at least one compound I.

An agrochemical composition comprises a pesticidally effective amount of a compound I.

The compounds I can be converted into customary types of agrochemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International. The compositions are prepared in a known manner, e.g. described by Mollet and Grubemann, Formulation technology, Wiley VCH, Weinheim, 2001; or Knowles, New developments in crop protection product formulation, Agrow Reports DS243, T&F Informa, London, 2005. Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders. Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emulsifier, dispersant, solubilizer, wetter, penetration enhancer, protective colloid, or adjuvant. Surfactants are listed in McCutcheon's, Vol.1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-subsituted fatty acid amides, amine oxides, esters, sugar-based surfactants, polymeric surfactants. Suitable cationic surfactants are qua-ternary surfactants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. Therefore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grape-fruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds. “Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aerosol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta Americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

The compounds of the invention are especially suitable for efficiently combating animal pests e.g. arthropods, and nematodes including:

insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;

Lepidoptera, e.g. Helicoverpa spp., Heliothis virescens, Lobesia botrana, Ostrinia nubilalis, Plutella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;

True bugs, e.g. Lygus spp., Stink bugs such as Euschistus spp., Halyomorpha halys, Nezara viridula, Piezodorus guildinii, Dichelops furcatus;

Thrips, e.g. Frankliniella spp., Thrips spp., Dichromothrips corbettii;

Aphids, e.g. Acyrthosiphon pisum, Aphis spp., Myzus persicae, Rhopalosiphum spp., Schizaphis graminum, Megoura viciae;

Whiteflies, e.g. Trialeurodes vaporariorum, Bemisia spp.;

Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;

Flies, e.g. Delia spp., Ceratitis capitate, Bactrocera spp., Liriomyza spp.;

Coccoidea, e.g. Aonidiella aurantia, Ferrisia virgate;

Anthropods of class Arachnida (Mites), e.g. Penthaleus major, Tetranychus spp.;

Nematodes, e.g. Heterodera glycines, Meloidogyne spp., Pratylenchus spp., Caenorhabditis elegans.

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound I.

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound I.

The compounds of the invention are further suitable for use in combating or controlling parasites in and on animals. Furthermore, the invention relates to a method of combating or controlling parasites in and on animals, which comprises contacting the parasites with a parasitically effective amount of a compound I.

The invention also relates to the non-therapeutic use of compounds I for controlling or combating parasites. Moreover, the invention relates to a non-therapeutic method of combating or controlling parasites, which comprises applying to a locus a parasiticidally effective amount of a compound I.

The compounds I can be effective through both contact (via soil, glass, wall, bed net, carpet, blankets or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides fells.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day.

Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition the compounds I may be transdermally administered to animals. For parenteral administration, the dosage form chosen should provide the animal with 0.01 mg/kg to 100 mg/kg of animal body weight per day of the compounds I.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds I. In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

### A. PREPARATION EXAMPLES

With appropriate modification of the starting materials, the procedures given in the synthesis description were used to obtain further compounds I. The compounds obtained in this manner are listed in the table that follows, together with physical data.

The products shown below were characterized by melting point determination, by NMR spectroscopy or by the masses ([m/z]) or retention time (RT; [min.]) determined by HPLC MS or HPLC spectrometry.

HPLC MS=high performance liquid chromatography-coupled mass spectrometry;

HPLC method A: Shimadzu Nexera UHPLC+Shimadzu LCMS 20-20, ESI; Column: Phenomenex Kinetex 1.7 μm XB-C18 100A, 50×2.1 mm; Mobile Phase: A: water+0.1% TFA; B: ACN; Temperature: 60° C.; Gradient: 5% B to 100% B in 1.50 min; 100% B 0.25 min; Flow: 0.8 mL/min to 1.0 mL/min in 1.51 min; MS method: ESI positive; Mass range (m/z): 100-700.

### Example 1: Preparation of N-[1-(2-pyrimidin-2-yl-1,2,4-triazol-3-yl)ethyl]-5,7-bis(trifluoromethyl)-1,2-benzoxazol-3-amine [I-1]

Step 1: A solution of 2-chloro-3,5-bis(trifluoromethyl)aniline (10 g, 0.038 mol) in MeCN (60 mL) was added slowly over 5 min into a solution of H₂O₄ (60 mL) and H₂O (60 mL) at 0° C., and stirred at that temperature for additional 10 min. NaNO₂ (4.6 g, 0.133 mol) in H₂O (40 mL) was added dropwise to the above mixture over 5 min, upon which the internal temperature reached 10° C., and stirred at that temperature for 10 min. A solution of KI (22 g, 66.5 mmol) in H₂O (60 mL) was added dropwise at 0° C. and stirred for 2 h. Then TLC analysis (PE, Rf=0.6) showed complete reaction. The organic phase was separated, and the aqueous phase was extracted with MTBE (2×50 mL). The combined organic phases were washed with NaHCO₃ solution (2×100 mL, sat. aq.), Na₂S₂O₃ solution (2×100 mL sat. aq.), dried over Na₂SO₄, filtered and concentrated under reduced pressure. Purification by column chromatography (PE) afforded 2-chloro-1-iodo-3,5-bis(trifluoromethyl)benzene (13 g, 93%) as a light brown oil. ¹H-NMR (CDCl₃, 400 MHz, RT): δ ppm 8.32 (s, 1H) 7.95 (s, 1H).

Step 2: To a solution of 2-chloro-1-iodo-3,5-bis(trifluoromethyl)benzene (12 g, 32 mmol) in NMP (50 mL) at 25° C. under a N₂ atmosphere was added CuCN (4.2 g, 48 mmol), and the resulting mixture was heated at 120° C. for 16 h. Then TLC analysis (PE/EtOAc=10:1, Rf=0.2) showed complete reaction. The reaction mixture was quenched with H₂O (200 mL), extracted with EtOAc (3×200 mL), and the combined organic extracts were dried over Na₂SO₄, filtered and concentrated under reduced pressure. Purification by column chromatography (PE/EtOAc=50:1, Rf=0.3) afforded 2-chloro-3,5-bis(trifluoromethyl)benzonitrile (7.0 g, 80%) as yellow solid. ¹H-NMR (CDCl₃, 400 MHz, RT): δ ppm 8.16 (br d, J=9.3Hz, 2H).

Step 3: To a solution of 2-chloro-3,5-bis(trifluoromethyl)benzonitrile (3.50 g, 12.8 mmol) in THF (100 mL) at 25° C. under a N₂ atmosphere was added t-BuOK (1.70 g, 15.3 mmol), and the resulting mixture was stirred at that temperature for 30 min. Propan-2-one oxime (CAS-No. 127-06-0; 1.12 g, 15.3 mmol) in THF (20 mL) was added to the above mixture and stirred at 25° C. for 2 h. Then TLC analysis (PE/EtOAc=10:1, Rf=0.5) showed complete reaction. The reaction mixture was quenched with NH₄Cl solution (100 mL, sat. aq.), extracted with EtOAc (2×100 mL), and the combined organic extracts were dried over Na₂SO₄, filtered and concentrated under reduced pressure to give crude 2-(isopropylideneamino)oxy-3,5-bis¬(trifluoromethyl)benzonitrile (3.0 g) as a brown oil, which was directly used in the next step without any further purification.

Step 4: To a solution of crude 2-(isopropylideneamino)oxy-3,5-bis(trifluoromethyl)benzonitrile (3.0 g, 9.7 mmol) in MeOH (10 mL) at 25° C. under a N₂ atmosphere was added HCl/MeOH (150 mL) and SOCl₂ (11.2 g, 9.49 mol), and the reaction mixture was stirred at that temperature for 72 h. Then TLC analysis (PE/EtOAc=5:1, Rf=0.4) showed complete reaction. The reaction mixture was concentrated, and the residue quenched with NaHCO₃ solution (200 mL, sat. aq.) to adjust the pH to 7. The aqueous phase was extracted with EtOAc (2×100 mL), and the combined organic extracts were dried over Na₂SO₄, filtered and concentrated under reduced pressure. Purification by column chromatography (PE/EtOAc=10:1, Rf=0.5) afforded 5,7-bis(trifluoromethyl)-1,2-benzoxazol-3-amine (2.0 g, 77%) as a red solid. ¹H-NMR (400 MHz, DMSO-d₆, RT): δ ppm 8.70 (s, 1H), 8.23 (s, 1H), 6.95 (s, 2H).

Step 5: To a solution of 5,7-bis(trifluoromethyl)-1,2-benzoxazol-3-amine (800 mg, 2.96 mmol) in toluene (10 mL) at 25° C. under a N₂ atmosphere was added Ti(OiPr)₄ (2.50 g, 8.88 mmol) and 1-(2-pyrimidin-2-yl-1,2,4-triazol-3-yl)ethanone (672 mg, 3.55 mmol, Example 8), before the mixture was heated at 100° C. for 16 h. Then LCMS showed complete reaction. Then, and the solution was used directly in the next step without further purification.

Step 6: To the solution obtained in Step 5 (ca. 10 mL in toluene) was added EtOH (20 mL) and the solution was cooled to 0° C. NaBH(OAc)₃ (1.25 g, 3.92 mmol) and Na(CN)BH₃ (372 mg, 3.92 mmol) were added slowly, and the reaction mixture was heated at 50° C. for 16 h. Then LCMS showed that the reaction was complete. The reaction was quenched with H₂O (100 mL), filtered and the filter cake was washed with EtOAC (2×50 mL). The aqueous phase was extracted with EtOAc (2×50 mL) and the combined organic phases were dried over Na₂SO₄, filtered, and concentrated under reduced pressure. Purification by preparative HPLC afforded N-[1-(2-pyrimidin-2-yl-1,2,4-triazol-3-yl)ethyl]-5,7-bis(trifluoromethyl)-1,2-benzoxazol-3-amine (220 mg, 34%) as a white solid.

LCMS (Method A): 1.14, 443.7

¹H-NMR (DMSO-d₆, 400 MHz, RT): δ ppm 9.01 (d, J=4.8Hz, 2H) 8.79 (s, 1 H) 8.21-8.28 (m, 2 H) 8.16 (s, 1H) 7.66 (t, J=4.8Hz, 1H) 5.79 (quin, J=7.1Hz, 1H) 1.73 (d, J=6.7Hz, 3H).

### Example 2: Preparation of N-[(2-pyrimidin-2-yl-1,2,4-triazol-3-yl)methyl]-5,7-bis(trifluoromethyl)-1,2-benzoxazol-3-amine [I-2]

Step 1: To a solution of 5,7-bis(trifluoromethyl)-1,2-benzoxazol-3-amine (1.00 g, 3.70 mmol; cf. Example 1) and ethyl 2-oxoacetate (567 mg, 5.55 mmol) in MeOH (10 mL) at 0° C. was added HSiEt₃ (1.29 g, 11.1 mmol) and F₃CCO₂H (1.27 g, 11.1 mmol), the cooling bath was removed, and the reaction was stirred at ambient temperature for 2 h. Then TLC-analysis (PE/EtOAc=5:1, Rf=0.7) showed the reaction was complete. The mixture was poured into NaHCO₃ solution (20 mL, sat. aq.), the aqueous phase was extracted with CH₂Cl₂ (3×5 mL), and the combined organic extracts were washed with NaCl solution (5 mL, sat. aq.), dried over Na₂SO₄, filtered, and concentrated under reduced pressure. Purification by silica gel column afforded ethyl 2-[[5,7-bis(trifluoromethyl)-1,2-benzoxazol-3-yl]amino]acetate (1.30 g, 98%) as a white solid.

¹H-NMR (400 MHz, CDCl₃, RT): δ ppm 8.08 (s, 1H), 8.00 (s, 1H), 5.16 (br s, 1H), 4.33 (q, J=7.1Hz, 2H), 4.23 (d, J=5.3Hz, 2H), 1.38-1.34 (m, 3H).

Step 2: To a solution of ethyl 2-[[5,7-bis(trifluoromethyl)-1,2-benzoxazol-3-yl]amino]acetate (1.30 g, 3.65 mmol) in a sealed pot was added NH₃, 7 M in MeOH (20 mL) at ambient temperature before the reaction mixture was heated to 70° C. and stirred at that temperature for 48 h. Then TLC analysis (EtOAc, Rf=0.7) showed the reaction was complete. The reaction mixture was concentrated under reduced pressure, the crude product was triturated with CH₂Cl₂ (8 mL), and filtered to afford 2-[[5,7-bis(trifluoromethyl)-1,2-benzoxazol-3-yl]amino]acetamide (600 mg, 50%) as a yellow solid.

¹H-NMR (400 MHz, DMSO-d₆, RT): δ ppm 8.93-8.76 (m, 1H), 8.27 (s, 1H), 7.82 (t, J=5.8Hz, 1H), 7.56 (br s, 1H), 7.17 (br s, 1H), 3.88 (d, J=6.0Hz, 2H).

Step 3: To a solution of 2-[[5,7-bis(trifluoromethyl)-1,2-benzoxazol-3-yl]amino]acetamide (300 mg, 0.916 mmol) in CH₂Cl₂ (3.0 mL) at ambient temperature was added N,N-dimethylformamide dimethyl acetal (218 mg, 1.83 mmol) before the reaction mixture was heated to 50° C. and stirred at that temperature for 3 h. Then TLC analysis (EtOAc, Rf=0.6) showed the reaction was complete. The mixture was concentrated under reduced pressure to afford 2-[[5,7-bis(trifluoromethyl)-1,2-benzoxazol-3-yl]amino]-N-(dimethylaminomethylene)acetamide (350 mg, crude) as yellow oil which was used directly in the next step without any further purification.

Step 4: To a solution of 2-[[5,7-bis(trifluoromethyl)-1,2-benzoxazol-3-yl]amino]-N-(dimethylaminomethylene)acetamide (350 mg, 0.92 mmol) in 1,4-dioxane (3.0 mL) at ambient temperature was added pyrimidin-2-ylhydrazine (101 mg, 0.92 mmol), after 5 min added AcOH (3.0 mL), and after additional 5 min the reaction mixture was heated to 80° C. and stirred at that temperature for 1.5 h. Then TLC (EtOAc=100%, Rf=0.5) and LCMS analysis showed the reaction was complete. The mixture was concentrated under reduced pressure, adjusted to pH=7 with NaHCO₃ solution (sat. aq.), the aqueous phase was extracted with EtOAc (3×20 mL), and the combined organic extracts were washed with NaCl solution (10 mL, sat. aq.), dried over Na₂SO₄, filtered, and concentrated. Purification by column chromatography on silica gel (EtOAc/PE 4:1) followed by trituration with CH₂Cl₂ (3 mL) afforded N-[(2-pyrimidin-2-yl-1,2,4-triazol-3-yl)methyl]-5,7-bis(trifluoromethyl)-1,2-benzoxazol-3-amine [I-2] (194 mg, 49%) as a yellow solid.

¹H-NMR (400 MHz, CDCl₃, RT): δ ppm 8.94 (d, J=4.8Hz, 2H), 8.18 (s, 1H), 8.12 (s, 1H), 8.00 (s, 1H), 7.43 (t, J=4.8Hz, 1H), 6.14 (br t, J=5.0Hz, 1H), 5.31 (d, J=5.1Hz, 2H).

### Example 3: Preparation of N-[1-(2-pyrimidin-2-yl-1,2,4-triazol-3-yl)ethyl]-5-(trifluoromethyl)isoxazolo[5,4-b]pyridin-3-amine [I-3]

Step 1: To a solution of 2-chloro-5-(trifluoromethyl)pyridine-3-carboxylic acid (2.26 g, 10 mmol) in THF (20 ml) was added Boc₂O (4.4 g, 20 mmol), NH₄HCO₃ (1.6 g, 20 mmol), pyridine (1.6 g, 20 mmol) and the mixture was stirred at 20° C. for 24 h. Then the resulting reaction mixture was concentrated, diluted with EtOAc (40 mL) and the organic phase was washed with H₂O (50 mL), NaCl solution (50 mL, sat. aq.), dried over Na₂SO₄, filtered, and concentrated under reduced pressure to give 2-chloro-5-(trifluoromethyl)pyridine-3-carboxamide (2 g, crude) as a yellow solid, which was used directly in the next step without further purification.

Step 2: To a solution of 2-chloro-5-(trifluoromethyl)pyridine-3-carboxamide (1.95 g, 8.7 mmol) in CH₂Cl₂ (40 mL) at 0° C. was added Et₃N (3.6 g, 34.8 mmol) and trifluoroacetic anhydride (3.6 g, 17.4 mmol, CAS-No. 407-25-0) dropwise, before the mixture was allowed to warm to 20° C. and stirred at that temperature for 4 h. The resulting mixture was poured into ice water (100 mL) and the aqueous phase was extracted with CH₂Cl₂ (2×50 mL). The combined organic extracts were washed with NaCl solution (2×50 mL), dried over Na₂SO₄, filtered, and concentrated under reduced pressure. Purification by column chromatography (PE/EtOAc=10:1) afforded 2-chloro-5-(trifluoromethyl)pyridine-3-carbonitrile (1.1 g, 53% over 2 steps) as yellow solid.

¹H-NMR (400 MHz, CDCl₃, RT) δ ppm 8.88 (d, J=1.6Hz, 1 H), 8.25 (d, J=2.4Hz, 1H).

Step 3: 2-Chloro-5-(trifluoromethyl)pyridine-3-carbonitrile (1 g, 5 mmol), ethanehydroxamic acid (0.45 g, 6 mmol), and K₂CO₃ (1.4 g, 10 mmol) were dissolved in H₂O (30 mL) at 20° C., and the mixture was heated at 60° C. for 6 h. Then the resulting solution was filtered and the filter cake collected to give 5-(trifluoromethyl)isoxazolo[5,4-b]pyridin-3-amine (0.8 g, 80%) as a white solid.

¹H-NMR (DMSO-d₆, 400 MHz, RT): δ ppm 8.95 (d, J=1.6Hz, 1H), 8.80 (d, J=1.9 Hz, 1H), 6.90 (s, 2H).

Step 4: To a solution of 5-(trifluoromethyl)isoxazolo[5,4-b]pyridin-3-amine (0.8 g, 4.0 mmol) and 1-(2-pyrimidin-2-yl-1,2,4-triazol-3-yl)ethanone (0.91 g, 4.8 mmol, prepared according to Example 1) in toluene (10 ml) at 20° C. was added Ti(OiPr)4 (2.25 g, 8 mol), and the mixture was heated at 100° C. for 16 h. Then the reaction temperature was reduced to 50° C., MeOH (3 mL) and NaBH₃(CN) (0.51 g, 8.0 mmol) were added, and stirring was continued at that temperature for 16 h. The reaction mixture was quenched by mixing with ice water (30 mL) and EtOAc (30 mL), followed by stirring for an additional 1 h at 20° C. The quenched reaction mixture was filtered through a pad of celite eluting with EtOAc (30 mL), and the aqueous phase was extracted with EtOAc (30 mL). The combined organic extracts were washed with NaCl solution (30 mL, sat. aq.), dried over Na₂SO₄, filtered, and concentrated under reduced pressure. Purification by prep-HPLC afforded N-[1-(2-pyrimidin-2-yl-1,2,4-triazol-3-yl)ethyl]-5-(trifluoromethyl)isoxazolo[5,4-b]pyridin-3-amine [I-3] (0.13 g, 9%) as a yellow solid.

¹H-NMR (400 MHz, CDCl₃, RT): δ ppm 8.94 (d, J=4.8Hz, 2H), 8.80 (d, J=1.5Hz, 1H), 8.27 (d, J=1.6Hz, 1H), 8.07 (s, 1H), 7.43 (t, J=4.8Hz, 1H), 6.32-6.20 (m, 1H), 6.03 (br d, J=8.9Hz, 1H), 1.79 (d, J=6.6Hz, 3H).

### Example 4: Preparation of 1-methyl-N-[1-(2-pyrimidin-2-yl-1,2,4-triazol-3-yl)ethyl]-5-(trifluoromethyl)indazol-3-amine [I-4]

Step 1: In an autoclave, NaOAc (3.5 g, 42.3 mmol) was added to a solution of methylhydrazine (6 g, 42.3 mmol) in n-BuOH (5 mL) at 25° C., and the resulting mixture was stirred at that temperature for 30 min. Then, 2-fluoro-5-(trifluoromethyl)benzonitrile (4 g, 21.1 mmol) was added, and the mixture was heated at 130° C. for 72 h. Then TLC (PE/EtOAc=3:1, Rf=0.2) showed that the reaction was complete. The reaction mixture was quenched with H₂O (50 mL), the aqueous phase was extracted with EtOAc (3×50 mL), and the combined organic extracts were dried over Na₂SO₄, filtered and concentrated under reduced pressure. Trituration with MTBE (100 mL), followed by filtration and collection of the filter cake afforded 1-methyl-5-(trifluoromethyl)indazol-3-amine (3 g, 67%) as a white oil.

¹H-NMR (CDCl₃, 400 MHz, RT): δ ppm 7.85 (s, 1H), 7.55 (dd, J=8.8, 0.9Hz, 1H), 7.28 (d, J=7.9 Hz, 1H), 4.00-4.28 (m, 2H), 3.89 (s, 3H).

Step 2: To a solution of 1-methyl-5-(trifluoromethyl)indazol-3-amine (400 mg, 1.86 mmol) in toluene (10 mL) at ambient temperature (RT, 20-25° C.) under a N₂ atmosphere was added Ti(O-iPr)₄ (1.5 g, 3.58 mmol), followed by 1-(2-pyrimidin-2-yl-1,2,4-triazol-3-yl)ethanone (422 mg, 2.83 mmol, prepared according to Example 1), and the mixture was heated to 100° C. for 16 h. Then LCMS showed completion of the reaction, and the mixture was used in the next step as toluene solution without any further purification.

Step 3: The above toluene solution (ca. 10 mL) was diluted with EtOH (10 mL), cooled to 0° C., NaBH(OAc)₃ (788 mg, 3.7 mmol) and Na(CN)BH₃ (233 mg, 3.7 mmol) were slowly added, and the reaction mixture then heated at 50° C. for 16 h. Then LCMS indicated completion of the reaction. The reaction mixture was quenched into H₂O (100 mL), filtered and the filter cake washed with EtOAc (2×50 mL). The aqueous phase was extracted with EtOAc (2×50 mL), and the combined organic extracts were dried over Na₂SO₄, filtered and concentrated under reduced pressure. Purification by preparative HPLC afforded 1-methyl-N-[1-(2-pyrimidin-2-yl-1,2,4-triazol-3-yl)ethyl] (trifluoromethyl)indazol-3-amine (200 mg, 37%) as a white solid.

¹H-NMR (DMSO-d₆, 400 MHz, RT): δ ppm 9.03 (d, J=4.9Hz, 2H), 8.25 (s, 1H), 8.07 (s, 1H), 7.67 (t, J=4.9 Hz, 1H), 7.46-7.52 (m, 1H), 7.38-7.44 (m, 1H), 7.08 (d, J=8.9Hz, 1H), 5.74-5.83 (m, 1H), 3.47 (s, 3H), 1.69 (d, J=6.9Hz, 3H).

### Example 5: Preparation of 5,7-dichloro-N-[1-(2-pyrimidin-2-yl-1,2,4-triazol-3-yl)ethyl]-1,2-benzothiazol-3-amine [I-5]

Step 1: To a solution of 2-amino-3,5-dichloro-benzonitrile (200 mg, 1.07 mmol) in MeCN (7 mL) at 0° C. was added a solution of tert-butylnitrite (220 mg, 2.14 mmol) and CuCl₂ (172 mg, 2.14 mmol) in MeCN (2 mL). The resulting reaction mixture was allowed to warm to 25° C. and stirred at that temperature for 16 h. Then TLC (PE/EtOAc=5:1, Rf=0.5) showed the reaction was completed. The reaction mixture was quenched with H₂O (20 mL), the aqueous phase was extracted with EtOAc (2×20 mL), and the combined organic extracts were washed with NaCl solution (20 mL, sat. aq.), dried over Na₂SO₄, filtered and concentrated under reduced pressure. Purification by preparative TLC afforded 2,3,5-trichlorobenzonitrile (176 mg, 88%) as a white solid.

¹H-NMR (CDCl₃, 400 MHz, RT): δ ppm 7.72 (d, J=2.4Hz, 1H), 7.60 (d, J=2.4Hz, 1H).

Step 2: To a solution of 2,3,5-trichlorobenzonitrile (600 mg, 2.9 mmol) in DMF (12 mL) at 25° C. was added Na₂S (336 mg, 4.36 mmol), and the resulting mixture was stirred at that temperature for 8 h. Then TLC (PE/EtOAc=5:1, Rf=0.5) showed that the reaction was complete. The reaction mixture was quenched into H₂O (20 mL), the pH adjusted to 3-4 with 2M HCl (10 mL), the aqueous phase extracted with EtOAc (2×20 mL), and the combined organic extracts were dried over Na₂SO₄, filtered and concentrated under reduced pressure. Purification by column chromatography (PE/EtOAc=5:1, Rf=0.3) afforded 3,5-dichloro-2-sulfanyl-benzonitrile (700 mg) as a yellow oil.

¹H-NMR (DMSO-d₆, 400 MHz, RT): δ ppm 7.29 (d, J=2.4Hz, 1H), 7.71 (d, J=2.4Hz, 1H).

Step 3: To a solution of 3,5-dichloro-2-sulfanyl-benzonitrile (2 g, 9.8 mmol) in NH₃·H₂O (60 mL) at 0° C. was added NaOH solution (ca. 3% in H₂O, 20 mL) and NaOCl solution (ca. 7% in H₂O, 6 mL). The resulting reaction mixture was allowed to warm to 20° C. and stirred at that temperature for 16 h. Then TLC (PE/EtOAc=5:1, Rf=0.2) showed that the reaction was complete. The reaction mixture was quenched into water (100 mL), extracted with EtOAc (2×50 mL), and the combined organic extracts were dried over Na₂SO₄, filtered and concentrated under reduced pressure. Purification by column chromatography (PE/EtOAc=5:1, Rf=0.3) afforded 5,7-dichloro-1,2-benzothiazol-3-amine (1.5 g, 75%) as a yellow solid.

¹H-NMR (DMSO-d₆, 400 MHz, RT): δ ppm 8.27 (d, J=2.0Hz, 1H), 7.88 (d, J=2.0Hz, 1H), 7.20 (d, J=2.0Hz, 2H).

Step 4: To a solution of 5,7-dichloro-1,2-benzothiazol-3-amine (500 mg, 2.3 mmol) in toluene (10 mL) at 25° C. under a N₂ atmosphere was added Ti(O-iPr)₄ (1.3 g, 4.6 mmol), followed by 1-(2-pyrimidin-2-yl-1,2,4-triazol-3-yl)ethanone (520 mg, 2.7 mmol, prepared according to Example 1), and the mixture was heated at 100° C. for 16 h. Then LCMS showed the reaction was complete. The reaction mixture was used the next step as toluene solution without any purification.

Step 5: The above toluene solution (ca. 10 mL) was diluted with EtOH (20 mL), cooled to 0° C., NaBH(OAc)₃ (970 mg, 4.6 mmol) and Na(CN)BH₃ (285 mg, 4.6 mmol) were added slowly, and the reaction mixture was heated at 50° C. for 16 h. Then LCMS showed that the reaction was completed. The reaction was quenched into H₂O (100 mL), filtered and the filter cake was washed with EtOAc (2×50 mL). The aqueous phase was extracted with EtOAc (2×50 mL), and the combined organic extracts were dried over Na₂SO₄, filtered and concentrated under reduced pressure. Purification by prep-HPLC afforded 5,7-dichloro-N-[1-(2-pyrimidin-2-yl-1,2,4-triazol-3-yl)ethyl]-1,2-benzothiazol-3-amine [I-5] (220 mg, 44%) as white solid.

¹H-NMR (DMSO-d₆, 400 MHz, RT): δ ppm 8.98 (d, J=4.8Hz, 2H), 8.41 (d, J=1.7Hz, 1H), 8.26 (d, J=8.0Hz, 1H), 8.11 (s, 1H), 7.82 (d, J=1.7Hz, 1H), 7.63 (t, J=4.9Hz, 1H), 5.99 (quin, J=7.1Hz, 1H), 1.70 (d, J=6.9Hz, 3H).

### Example 6: Preparation of 8-chloro-N-(cyclopropylmethyl)-N-[1-(2-pyrimidin-2-yl-1,2,4-triazol-3-yl)ethyl]-6-(trifluoromethyl)-[1,2,4]triazolo[4,3-a]pyridin-3-amine [I-6]

Step 1: To a solution of 2,3-dichloro-5-(trifluoromethyl)pyridine (5.00 g, 23.2 mmol) in EtOH (50 mL) was added N₂H₄·H₂O (2.36 g, 46.3 mmol) and the reaction mixture was stirred for 6 h at 90° C. Then TLC analysis (PE/EtOAc=1:1) showed the reaction was complete. The reaction mixture was concentrated, the residue extracted with EtOAc (3×50 mL), and the combined organic extracts were washed with NaCl solution (30 mL, sat. aq.), dried over Na₂SO₄, filtered and concentrated under reduced pressure to give [3-chloro-5-(trifluoromethyl)-2-pyridyl]hydrazine (4.6 g, 94%) as white solid.

¹H-NMR (DMSO-d₆, 400 MHz, RT): δ ppm 4.47 (s, 2H), 7.91 (d, J=2.0Hz, 1H), 8.36 (d, J=0.86Hz, 1H), 8.52 (s, 1H).

Step 2: To a solution of [3-chloro-5-(trifluoromethyl)-2-pyridyl]hydrazine (3.85 g, 18.3 mmol) in EtOH/H₂O (77 mL/15.4 mL) was added BrCN (2.9 g, 27.4 mmol) in EtOH/H₂O (5.8 mL/1.2 mL) dropwise, and the mixture was stirred at 20° C. for 6 h. Then TLC (PE/EtOAc=1:1) showed that the reaction was complete. The reaction mixture was concentrated, the residue quenched with H₂O (20 mL), the aqueous phase extracted with EtOAc (2×30 mL), and the combined organic phases were washed with NaCl solution (30 mL, sat. aq.), dried over Na₂SO₄, and concentrated under reduced pressure. Purification by silica gel column chromatography (PE/EtOAc=100:0 to 45:55, gradient) afforded 8-chloro-6-(trifluoromethyl)-[1,2,4]triazolo[4,3-a]pyridin-3-amine (2.2 g, 51%) as yellow solid. ¹H-NMR (DMSO-d₆, 400 MHz, RT): δ ppm 6.84-6.90 (m, 2H), 7.50-7.54 (m, 1H), 8.77-8.80 (m, 1H).

Step 3: To a solution of 8-chloro-6-(trifluoromethyl)-[1,2,4]triazolo[4,3-a]pyridin-3-amine (1 g, 4.2 mmol) and 1-(2-pyrimidin-2-yl-1,2,4-triazol-3-yl)ethanone (800 mg, 4.2 mmol, prepared according to Example 1) in toluene (20 mL) was added Ti(OiPr)₄ (0.91 g, 3.2 mmol) and the mixture was heated at 110° C. for 4 h. Then TLC analysis (PE/EtOAc=1:1) showed that the reaction was complete. The mixture was cooled to 20-25° C., EtOH (10 mL) and NaBH₃CN (2.7 g, 42 mmol) were added, and the resulting mixture was stirred at 40° C. for 16 h, then TLC analysis (DCM/MeOH=10:1) showed the reaction was complete. The reaction mixture was quenched with H₂O (15 mL), filtered, the filtrate was extracted with EtOAc (3×10 mL), and the combined organic phases were washed with NaCl solution (20 mL, sat. aq.), dried over Na₂SO₄, and concentrated under reduced pressure. Purification by silica gel column chromatography (DCM/MeOH=100:0 to 12:88, gradient) to give 8-chloro-N-[1-(2-pyrimidin-2-yl-1,2,4-triazol-3-yl)ethyl]-6-(trifluoromethyl)-[1,2,4]triazolo[4,3-a]pyridin-3-amine (1.3 g, 75%).

¹H-NMR (CDCl₃, 400 MHz, RT): δ ppm 1.81 (d, J=6.8Hz, 3H), 6.32 (d, J=9.4Hz, 1H), 6.44 (dd, J=9.38, 6.8Hz, 1H), 7.20 (d, J=1.0Hz, 1H), 7.40 (t, J=4.9Hz, 1H), 8.01 (s, 1H), 8.18 (s, 1H) 8.90 (d, J=4.8Hz, 2H).

Step 4: To a solution of 8-chloro-N-[1-(2-pyrimidin-2-yl-1,2,4-triazol-3-yl)ethyl]-6-(trifluoromethyl)-[1,2,4]triazolo[4,3-a]pyridin-3-amine (1.3 g, 3.2 mmol) in DMF (13 mL) at 0° C. was added NaH (4.8 g, 4.8 mmol) portion wise, and the mixture was stirred at that temperature for 0.5 h. (bromomethyl)cyclopropane (0.8 mL, 6.4 mmol) was added, and the resulting mixture was allowed to slowly warm to ambient temperature over 4 h. Then TLC analysis (DCM/MeOH=10:1) showed that the reaction was complete. The reaction mixture was quenched with NH₄Cl solution (8 mL, sat. aq.), extracted with EtOAc (3×10 mL), and the combined organic extracts were washed with NaCl solution (15 mL, sat. aq.), dried over Na₂SO₄, filtered, and concentrated under reduced pressure. Purification by preparative HPLC afforded the title 8-chloro-N-(cyclopropylmethyl)-N-[1-(2-pyrimidin-2-yl-1,2,4-triazol-3-yl)ethyl]-6-(trifluoromethyl)-[1,2,4]triazolo[4,3-a]pyridin-3-amine [I-6] (210 mg, 14%) as a yellow solid.

¹H-NMR (CDCl₃, 400 MHz, RT): δ ppm 8.52 (br s,1H), 8.36 (br d, J=4.4Hz, 2H), 7.94 (s, 1H), 7.43 (s, 1H), 6.81 (t, J=4.8Hz, 1H), 6.34-6.50 (m, 1H), 3.82 (br d, J=7.0Hz, 2H), 1.89 (br d, J=7.1Hz, 3H), 1.11 (br dd, J=5.9, 2.2Hz, 1H), 0.47-0.69 (m, 2H), 0.25 (br d, J=3.6 Hz, 2H).

### Example 7: Preparation of 2-methyl-5-(1,1,2,2,2-pentafluoroethyl)-N-[1-(2-pyrimidin-2-yl-1,2,4-triazol-3-yl)ethyl]-4-(trifluoromethyl)pyrazol-3-amine [I-7]

Step 1: To a solution of 5-fluoro-1-methyl-3-(1,1,2,2,2-pentafluoroethyl)-4-(trifluoromethyl)pyrazole (75.2 mg, 0.263 mmol, CAS-No. 104315-28-8) and 1-(2-pyrimidin-2-yl-1,2,4-triazol-3-yl)ethanamine (50.0 mg, 0.263 mmol, prepared according to WO2017/192385) in MeCN (3.0 mL) at ambient temperature was added Cs₂CO₃ (171 mg, 0.525 mmol), and the resulting reaction mixture was heated at 65° C. for 10 h. Then the reaction mixture was concentrated under reduced pressure and the residue purified by column chromatography (CyH/EtOAc 100:0 to 0:100, gradient) to give the title 2-methyl-5-(1,1,2,2,2-pentafluoroethyl)-N-[1-(2-pyrimidin-2-yl-1,2,4-triazol-3-yl)ethyl]-4-(trifluoromethyl)pyrazol-3-amine [I-7] (28.0 mg, 22%) as a slightly yellowish oil.

¹H-NMR (CDCl₃, 400 MHz, RT): δ ppm 8.30 (s, 1H), 8.29 (s, 2H), 6.58 (t, J=4.8Hz, 1H), 5.90 (d, J=8.2Hz, 1H), 5.49 (dq, J=8.3, 6.9Hz, 1H), 3.78 (s, 3H), 1.70 (d, J=6.9 Hz, 3H).

### Example 8: Preparation of 1-(2-pyrimidin-2-yl-1,2,4-triazol-3-yl)ethenone

Step 1: To a solution of 2-hydroxypropanamide (7 g, 79 mmol) in CH₂Cl₂ (100 mL) at 25° C. under a N₂ atmosphere was added dimethylformamide dimethylacetal (24 g, 200 mmol, CAS-No. 4637-24-5), and the reaction mixture was heated at 50° C. for 2 h. Then TLC analysis (EtOAc, Rf=0.1) showed that the reaction was complete, and the resulting solution was concentrated to give crude N-(dimethylaminomethylene)-2-hydroxy-propanamide (12 g) as a yellow oil, which was used in the next step without further purification.

Step 2: To a solution of N-(dimethylaminomethylene)-2-hydroxy-propanamide (7 g, 63 mmol) in a mixture of 1,4-dioxane (90 mL) and AcOH (90 mL) at 90° C. was added a solution of pyrimidin-2-ylhydrazine (12 g, 85 mmol) in 1,4-dioxane (90 mL) dropwise, and stirring was continued at 90° C. for 2 h. Then TLC (PE/EtOAc=3:1, Rf=0.4) showed the reaction was complete. The resulting reaction mixture was concentrated under reduced pressure, and the residue quenched with Na-HCO₃ solution (200 mL, sat. aq.). The aqueous phase was extracted with CH₂Cl₂/i-PrOH (3:1, 3×100 mL), and the combined organic extracts dried over Na₂SO₄, filtered, and concentrated under reduced pressure. Purification by column chromatography (EtOH/EtOAc=1:3, Rf=0.3) afforded 1-(2-pyrimidin-2-yl-1,2,4-triazol-3-yl)ethanol (5.2 g, 35%) as yellow solid.

¹H-NMR (CDCl₃, 400 MHz, RT): δ ppm 8.92 (d, J=4.9Hz, 2H), 8.05 (s, 1H), 7.44 (t, J=4.9Hz, 1H), 5.24-5.37 (m, 1H), 5.18 (d, J=5.1Hz, 1H), 1.76 (d, J=6.7Hz, 3H).

Step 3: To a solution of 1-(2-pyrimidin-2-yl-1,2,4-triazol-3-yl)ethanol (5.2 g, 27.2 mmol) in CH₂Cl₂ (200 mL) at 0° C. was added Dess-Martin-Periodinane (CAS-No. 87413-09-0; 17 g, 42.8 mmol) slowly under N₂, before the reaction mixture was allowed to warm to 25° C. and stirred at that temperature for 16 h. Then TLC analysis (EtOAc, Rf=0.4) showed that the reaction was complete. The resulting solution was quenched into H₂O (100 mL), extracted with DCM (3×100 mL), and the combined organic extracts were washed with NaHCO₃ solution (2×200 mL), dried over Na₂SO₄, filtered and concentrated under reduced pressure. Purification by column chromatography (PE/EtOAc=1:1, Rf=0.23) afforded 1-(2-pyrimidin-2-yl-1,2,4-triazol-3-yl)ethenone (3.5 g, 69% yield) as a yellow solid.

¹H-NMR (CDCl₃, 400 MHz, RT): δ ppm 8.85 (d, J=4.9Hz, 2H), 8.13 (s, 1H), 7.45 (t, J=4.9Hz, 1H), 2.76 (s, 3H).

### Example 9: Preparation of 5,7-dichloro-1-oxo-N-[1-(2-pyrimidin-2-yl-1,2,4-triazol-3-yl)ethyl]-1,2-benzothiazol-3-amine [I-8] and 5,7-dichloro-1,1-dioxo-N-[1-(2-pyrimidin-2-yl-1,2,4-triazol-3-yl)ethyl]-1,2-benzothiazol-3-amine [I-9]

Step 1: To a solution of 5,7-dichloro-N-[1-(2-pyrimidin-2-yl-1,2,4-triazol-3-yl)ethyl]-1,2-benzothiazol-3-amine [I-5] (900 mg, 2.6 mmol) in CH₂Cl₂ (10 mL) at ambient temperature was added meta-chloroperoxybenzoic acid (660 mg, 3.9 mmol) and stirring was continued at that temperature for 20 h. After that time, the reaction mixture was quenched with H₂O (100 mL), the aqueous phase extracted with DCM/i-PrOH 3:1 (3×100 mL), and the combined organic extracts were dried over Na₂SO₄, filtered, and concentrated.

Purification by preparative HPLC afforded 5,7-dichloro-1,1-dioxo-N-[1-(2-pyrimidin-2-yl-1,2,4-triazol-3-yl)ethyl]-1,2-benzothiazol-3-amine [I-9] (116 mg, 11%) as a yellow solid and 5,7-dichloro-1-oxo-N-[1-(2-pyrimidin-2-yl-1,2,4-triazol-3-yl)ethyl]-1,2-benzothiazol-3-amine [I-8] (65 mg, 6.1%) as yellow solid.

[I-9]: ¹H-NMR (400 MHz, DMSO-d₆, RT): δ ppm 8.92 (d, J=4.8Hz, 2H), 8.23 (s, 1H), 8.18 (br s, 1H), 8.06 (d, J=1.2Hz, 1H), 7.59 (t, J=4.8Hz, 1H), 6.04 (q, J=6.9Hz, 1H), 1.69 (d, J=6.8Hz, 3H).

[I-8]: ¹H-NMR (400 MHz, DMSO-d₆, RT): δ ppm 9.58-9.45 (m, 1H), 9.02-8.93 (m, 2H), 8.42-8.34 (m, 1H), 8.25-8.18 (m, 1H), 7.98 (d, J=1.3Hz, 1H), 7.67-7.58 (m, 1H), 6.26-6.10 (m, 1H), 1.78-1.67 (m, 3H).

### Example 10: tert-butyl 2-[[5,7-bis(trifluoromethyl)-1,2-benzoxazol-3-yl]-methoxycarbonyl-amino]propanoate

Step 1: To a solution of tert-butyl 2-[[5,7-bis(trifluoromethyl)-1,2-benzoxazol-3-yl]-methoxycarbonyl-amino]-acetate (550 mg, 1.24 mmol, cf. Example 15) in CH₂Cl₂ (16 mL) at −60° C. under a N₂ atmosphere was added potassium bis(trimethylsilyl)amide (1.86 mL, 1.86 mmol), followed after 0.5 h by the dropwise addition of MeOTf (408 mg, 2.49 mmol). The resulting reaction mixture was stirred between −60° C. to 0° C. for 12 h, then TLC analysis (PE/EtOAc=5:1) showed the reaction was complete. The reaction mixture was poured into an ice cooled NH₄Cl solution (100 mL, sat. aq.), the aqueous phase was extracted with CH₂Cl₂ (3×50 mL), and the combined organic extracts were washed with NaCl solution (sat. aq.), dried over Na₂SO₄, filtrated and concentrated under reduced pressure. Purification by column chromatography on silica gel (EtOAc/PE 0:100 to 7:93, gradient) afforded tert-butyl 2-[[5,7-bis(trifluoromethyl)-1,2-benzoxazol-3-yl]-methoxycarbonyl-amino]propanoate contaminated with starting material (450 mg).

¹H-NMR (400 MHz, CDCl₃, RT): δ ppm 8.33 (s, 1H), 8.05 (s, 1H), 4.99 (q, J=7.15Hz, 1H), 3.87 (s, 3H), 1.63 (d, J=7.28Hz, 3H), 1.42 (s, 9H).

The above tert-butyl 2-[[5,7-bis(trifluoromethyl)-1,2-benzoxazol-3-yl]-methoxycarbonylamino]propanoate could, after acidic Boc-cleavage, then be further elaborated into e.g. compound 1-36 analogously as described in Example 11.

### Example 11: Preparation of N-[1-(2-pyrimidin-2-yl-1,2,4-triazol-3-yl)propyl]-5,7-bis(trifluoromethyl)-1,2-benzoxazol-3-amine [I-28]

Step 1: To a solution of 5,7-bis(trifluoromethyl)-1,2-benzoxazol-3-amine (270 mg, 1.00 mmol; cf. Example 1) and ethyl 2-oxobutanoate (195 mg, 1.50 mmol) in 1,2-dichloroethane (3.0 mL) at ambient temperature was added Et₃SiH (349 mg, 3.00 mmol) and F₃CCO₂H (342 mg, 11.1 mmol) before the reaction mixture was heated to 80° C. and stirred at that temperature for 36 h. Then TLC analysis (PE/EtOAc=5:1, Rf=0.6) showed the reaction was complete. The mixture was poured into H₂O (10 mL), the aqueous phase extracted with CH₂Cl₂ (3×5 mL), and the combined organic extracts were washed with NaCl solution (5 mL, sat. aq.), dried over Na₂SO₄, filtered, and concentrated under reduced pressure. Purification by silica gel column (EtOAc in PE=7%) afforded ethyl 2-[[5,7-bis(trifluoromethyl)-1,2-benzoxazol-3-yl]amino]butanoate (300 mg, 86%) as a white solid.

¹H-NMR (400 MHz, CDCl₃, RT): δ ppm 8.06 (s, 1H), 7.96 (s, 1H), 5.40 (br d, J=7.6 Hz, 1H), 4.60-4.42 (m, 1H), 4.39-4.21 (m, 2H), 2.21-2.08 (m, 1H), 1.93 (qd, J=7.2, 14.0Hz, 1H), 1.36 (t, J=7.1Hz, 3H), 1.03 (t, J=7.4Hz, 3H).

Step 2: To a solution of ethyl 2-[[5,7-bis(trifluoromethyl)-1,2-benzoxazol-3-yl]amino]butanoate (1.5 g, 3.91 mmol) in THF (20 mL) at 0° C. was added LiOHH₂O (328 mg, 7.80 mmol) in H₂O (10 mL), before the reaction mixture was allowed to warm to ambient temperature and stirred at that temperature for 3 h. Then TLC analysis (PE/EtOAc=1:1, Rf=0.5) showed the reaction was complete. The mixture was poured into H₂O (20 mL), the aqueous phase extracted with EtOAc (3×30 mL), and the combined organic extracts were washed with NaCl solution (10 mL, sat. aq.), dried over Na₂SO₄, filtered, and concentrated under reduced pressure. Purification by column chromatography on silica gel (PE/EtOH=3:1) afforded 2-[[5,7-bis(trifluoromethyl)-1,2-benzoxazol-3-yl]amino]butanoic acid (1.2 g, 86%) as a yellow solid.

¹H-NMR (400 MHz, DMSO-d₆, RT): δ ppm 8.96 (s, 1H), 8.32-8.19 (m, 1H), 7.95 (s, 1H), 7.71 (br d, J=8.0Hz, 1H), 4.09-3.94 (m, 1H), 2.01-1.70 (m, 2H), 0.99 (t, J=7.4Hz, 3H)

Step 3: To a solution of 2[[5,7-bis(trifluoromethyl)-1,2-benzoxazol-3-yl]amino]butanoic acid (1.20 g, 3.40 mmol) in THF (10 mL) at ambient temperature was added Boc2O (1.46 g, 6.80 mmol), pyridine (529 mg, 6.80 mmol) and NH₄HCO₃ (529.3 mg, 6.8 mmol), and the reaction mixture was stirred at that temperature for 16 h. Then TLC analysis (PE/EtOAc=1:1, Rf=0.4) showed the reaction was complete. The mixture was concentrated under reduced pressure and purified by column chromatography on silica gel (EtOAc in PE=70%) to give 2-[[5,7-bis(trifluoromethyl)-1,2-benzoxazol-3-yl]amino]butanamide (1.0 g, 83%) as a yellow solid.

¹H-NMR (400 MHz, DMSO-d₆, RT): δ ppm 8.93 (s, 1H), 8.30-8.18 (m, 1H), 7.65-7.55 (m, 2H), 7.11 (s, 1H), 4.37 (t, J=5.1Hz, 1H), 1.92-1.67 (m, 2H), 1.17 (t, J=7.1Hz, 3H).

Step 4: To a solution of 2-[[5,7-bis(trifluoromethyl)-1,2-benzoxazol-3-yl]amino]butanamide (1.0 g, 2.8 mmol) in CH₂Cl₂ (10 mL) at ambient temperature was added N,N-dimethylformamide dimethyl acetal (672 mg, 5.64 mmol) before the resulting reaction mixture was heated to 50° C. and stirred at that temperature for 2 h. Then TLC analysis (EtOAc, Rf=0.6) showed the reaction was complete. The mixture was concentrated under reduced pressure to give 2-[[5,7-bis(trifluoromethyl)-1,2-benzoxazol-3-yl]amino]-N-(dimethylaminomethylene)butanamide (1.2 g, crude) as a yellow oil, which was used directly in the next step without any further purification.

Step 5: To a solution of 2-[[5,7-bis(trifluoromethyl)-1,2-benzoxazol-3-yl]amino]-N-(dimethylaminomethylene)butanamide (1.2 g, 2.9 mmol) in 1,4-dioxane (4.0 mL) at ambient temperature was added pyrimidin-2-ylhydrazine (321 mg, 2.92 mmol) and, after stirring for additional 5 min, AcOH (4 mL), before the reaction mixture was heated to 80° C. and stirred at that temperature for 1.5 h. Then the mixture was concentrated under reduced pressure, adjusted the pH =7 with Na—HCO₃ (sat. aq.), extracted the aqueous phase with EtOAc (3×30 mL), and the combined organic extracts were washed with NaCl solution (10 mL), dried over Na₂SO₄, filtered and concentrated. Purification by preparative HPLC (TFA) afforded N-[1-(2-pyrimidin-2-yl-1,2,4-triazol-3-yl)propyl]-5,7-bis(trifluoromethyl)-1,2-benzoxazol-3-amine [I-28] (441 mg, 33%) as a white solid.

¹H-NMR (400 MHz, CDCl₃, RT): δ ppm 8.97 (d, J=4.9Hz, 2H), 8.14 (s, 2H), 7.93 (s, 1H), 7.49 (t, J=4.8Hz, 1H), 6.68 (br d, J=9.7Hz, 1H), 6.23 (dt, J=4.9, 8.8Hz, 1H), 2.32-2.21 (m, 1H), 2.11 (quint, J=7.4, 14.6Hz, 1H), 1.17 (t, J=7.4Hz, 3H).

### Example 12: Preparation of 2-[[5,7-bis(trifluoromethyl)-1,2-benzoxazol-3-yl]-[1-(2-pyrimidin-2-yl-1,2,4-triazol-3-yl)ethyl]amino]ethyl benzoate [I-39]

Preparation of 2-[1-(2-pyrimidin-2-yl-1,2,4-triazol-3-yl)ethylamino]ethyl benzoate:

Step 1: To a mixture of (2,2-dimethyl-1,3-dioxolan-4-yl)methanol (100 g, 0.76 mol) and Et₃N (76 g, 0.76 mol) in CH₂Cl₂ (1.0 L) at 0° C. under a N₂ atmosphere was added PhC(O)Cl (106 g, 0.76 mol) dropwise, the cooling bath was removed, and the reaction mixture was stirred at ambient temperature for 6 h. Then the organic phase was washed with H₂O (1 L) and NaCl solution (1L, sat. aq.), dried over Na₂SO₄, filtered, and concentrated under reduced pressure. The residue was re-dissolved in MeOH (1 L), Amberlyst-15 (100 g) was added at ambient temperature, and the resulting mixture was stirred at that temperature for 16 h. TLC analysis (PE/EtOAc=1:1, Rf=0.4) showed the reaction was complete. The reaction mixture was filtered, and the filtrate was concentrated to afford 2,3-dihydroxypropyl benzoate (110 g, crude) as a colorless oil, which could be used in the next step without further purification.

¹H-NMR (400 MHz, CDCl₃, RT): δ ppm 8.11-8.00 (m, 2H), 7.62-7.52 (m, 1H), 7.50-7.40 (m, 2H), 4.51-4.33 (m, 2H), 4.12-4.05 (m, 1H), 3.86-3.64 (m, 2H).

Step 2: To a mixture of 2,3-dihydroxypropyl benzoate (65 g, 0.33 mol) and NalO₄ (141 g, 0.66 mol) in CH₂Cl₂ (600 mL) at ambient temperature was added NaHCO₃ (30 mL, sat. aq.), and the resulting reaction mixture was stirred at that temperature for 6 h. Then TLC analysis (PE/EtOAc=1:1, Rf=0.4) showed the reaction was complete. The mixture was dried over Na₂SO₄ and concentrated to give 2-oxoethyl benzoate (55 g, crude) as colorless oil, which could be used directly in the next step without further purification.

Step 3: To a solution of 1-(2-pyrimidin-2-yl-1,2,4-triazol-3-yl)ethanamine (45 g, 0.20mo1, prepared as described in WO2017/192385) in MeOH (500 mL) at ambient temperature was added Et₃N (20 g, 0.20 mol), 2-oxoethyl benzoate (49 g, 0.30 mol), and 4A MS (100 g). After 10 min, NaCNBH₃ (37 g, 0.6 mol) was added in small potions and stirring was continued at that temperature for 16 h. Then TLC analysis (EtOAc/EtOH=3:1, Rf=0.5) showed the reaction was complete. The mixture was filtered, and the filtrate was concentrated under reduced pressure. Purification by column chromatography (EtOH/EtOAc=30%) and preparative HPLC (NH₄HCO₃) afforded 2-[1-(2-pyrimidin-2-yl-1,2,4-triazol-3-yl)ethylamino]ethyl benzoate (21 g, 31%) as a colorless oil.

¹H-NMR (400 MHz, DMSO-d₆, RT): δ ppm 8.95 (d, J=4.9Hz, 2H), 8.15 (s, 1H), 7.91-7.84 (m, 2H), 7.66-7.55 (m, 2H), 7.53-7.42 (m, 2H), 4.74 (q, J=6.6Hz, 1H), 4.25-4.11 (m, 2H), 2.84-2.65 (m, 2H), 2.59 (br s, 1H), 1.43 (d, J=6.8Hz, 3H).

### Preparation of 2-[[5,7-bis(trifluoromethyl)-1,2-benzoxazol-3-yl]-[1-(2-pyrimidin-2-yl-1,2,4-triazol-3-yl)ethyl]amino]ethyl benzoate [I-39]

Step 1: A mixture of 2-chloro-1-iodo-3,5-bis(trifluoromethyl)benzene (50 g, 0.13mol), Et₃N (27 g, 0.27 mol), Pd(OAc)₂ (1.0 g, cat.), and 1,1′-bis(diphenylphosphino)ferrocene (1.0 g, cat.) in MeOH (300 mL)/MeCN (1.2 L), under a CO atmosphere (gas, 50 psi) was heated to 50° C. and stirred at that temperature for 12 h. Then TLC analysis (PE, Rf=0.4) showed the reaction was complete. The mixture was concentrated under reduced pressure and purified by column chromatography (PE) to give methyl 2-chloro-3,5-bis(trifluoromethyl)benzoate (37 g, 89%) as a yellow oil. ¹H-NMR (400 MHz, DMSO-d₆, RT): δ ppm 8.47 (s, 1H), 8.35 (s, 1H), 3.93 (s, 3H).

Step 2: To a solution of methyl 2-chloro-3,5-bis(trifluoromethyl)benzoate (110 g, 0.36 mmol) in THF (1.0 L) at ambient temperature and under a N₂ atmosphere was added NaOMe (29 g, 0.54 mol) in one potion, and the resulting reaction mixture was stirred at that temperature for 16 h. Then TLC analysis (PE/EtOAc=10:1, Rf=0.6) showed the reaction was complete. The mixture was concentrated, the residue was diluted with NH₄Cl solution (1 L, sat. aq.), the aqueous phase was extracted with EtOAc (2×500 mL), and the combined organic extracts were washed with NaCl solution (1 L, sat. aq.), dried over Na₂SO₄, filtered, and concentrated under reduced pressure. Purification by column chromatography (EtOAc/PE 5:95) afforded methyl 2-methoxy-3,5-bis(trifluoromethyl)benzoate (100 g, 92%) as colorless oil.

¹H-NMR (400 MHz, CDCl₃, RT): δ ppm 8.30 (d, J=2.1Hz, 1H), 8.01 (d, J=2.3Hz, 1H), 4.00 (s, 3H), 3.99 (s, 3H).

Step 3: To a solution of methyl 2-methoxy-3,5-bis(trifluoromethyl)benzoate (100 g, 0.33 mmol) in CH₂Cl₂ (1.0 L), at ambient temperature and under a N₂ atmosphere, was added BBr₃ (124 g, 0.50 mol) dropwise and the resulting reaction mixture was stirred at that temperature for 2 h. Then TLC analysis (EtOAc/PE 10:90, Rf=0.4) showed the reaction was complete. The mixture was poured into ice water (1.5 L), the aqueous phase was extracted with CH₂Cl₂ (2×500 mL), and the combined organic extracts were washed with NaCl solution (1 L, sat. aq.), dried over Na₂SO₄, filtered, and concentrated under reduced pressure. Purification by column chromatography (EtOAc/PE 10:90) afforded methyl 2-hydroxy-3,5-bis(trifluoromethyl)benzoate (67 g, 70%) as a white solid.

¹H-NMR (400 MHz, CDCl₃, RT): δ ppm 11.9 (s, 1H), 8.33 (s, 1H), 8.01 (s, 1H), 4.08-4.03 (m, 3H).

Step 4: To a solution of methyl 2-hydroxy-3,5-bis(trifluoromethyl)benzoate (26 g, 90 mmol) in THF (300 mL) at ambient temperature was added NaBH₄ (10 g, 270 mmol) in portions before the reaction mixture was heated to 60° C. and stirred at that temperature for 16 h. Then TLC analysis (PE/EtOAc 10:1, Rf=0.2) showed the reaction was complete. The mixture was poured into NH₄Cl solution (500 mL, sat. aq.), the aqueous phase was extracted with EtOAc (500 mL), and the organic extracts were washed with NaCl solution (500 mL, sat. aq.), dried over Na₂SO₄, filtered, and concentrated under reduced pressure. Purification by column chromatography (EtOAc/PE 30:70) afforded 2-(hydroxymethyl)-4,6-bis(trifluoromethyl)phenol (15.5 g, 66%) as white solid.

Step 5: To a mixture of 2-(hydroxymethyl)-4,6-bis(trifluoromethyl)phenol (31 g, 0.12mol) in CH₂Cl₂ (300 mL) at 0° C. was added DMP (101 g, 0.24mo1; CAS-No. 87413-09-0) in portions before the cooling bath was removed and the reaction mixture stirred at ambient temperature for 4 h. Then TLC analysis (PE/EtOAc=10:1, Rf=0.5) showed the reaction was complete. The mixture was poured into Na₂SO₃ (1.5 L, sat. aq.), the aqueous phase extracted with CH₂Cl₂ (2×500 mL), and the combined organic extracts were washed with NaCl solution (500 mL, sat. aq.), dried over Na₂SO₄, filtered, and concentrated under reduced pressure. Purification by column chromatography (EtOAc/PE 20:80) afforded 2-hydroxy-3,5-bis(trifluoromethyl)benzaldehyde (14 g, 46%) as yellow oil.

¹H-NMR (400 MHz, CDCl₃, RT): δ ppm 12.1 (s, 1H), 10.0 (s, 1H), 8.07 (s, 2H).

Step 6: To a mixture of 2-hydroxy-3,5-bis(trifluoromethyl)benzaldehyde (14 g, 54 mmol) and HONH₂·HCl (4.5 g, 65 mmol) in EtOH (14 mL) at ambient temperature was added NaOAc (6.6 g, 81 mmol) and the resulting reaction mixture was stirred at that temperature for 2 h. Then TLC analysis (PE/EtOAc=10:1, Rf=0.4) showed that the reaction was complete. The mixture was concentrated under reduced pressure, the residue was diluted with H₂O (200 mL), the aqueous phase was extracted with EtOAc (2×100 mL), and the combined organic extracts were washed with NaCl solution (1 L, sat. aq.), dried over Na₂SO₄, filtered, and concentrated. Purification by column chromatography (EtOAc/PE 25:75) afforded 2-hydroxy-3,5-bis(trifluoromethyl)benzaldehyde oxime (7.0 g, 47%) as a yellow solid.

¹H-NMR (400 MHz, DMSO-d₆, RT): δ ppm 12.2 (brs, 1H), 12.0 (brs, 1H), 8.60 (s, 1H), 8.16 (d, J=1.3Hz, 1H), 7.88 (s, 1H).

Step 7: To a solution of 2-hydroxy-3,5-bis(trifluoromethyl)benzaldehyde oxime (273 mg, 1.0 mmol) in DMF (3.0 mL) at 15° C. under a N₂ atmosphere was added N-chlorosuccinimide (266 mg, 2.0 mmol) and the resulting reaction mixture was stirred at that temperature for 2 h, before it was heated at 35° C. for an additional 0.5 h. Then TLC analysis (PE/EtOAc=10:1) showed that the reaction was complete. The mixture was poured into H₂O (10 mL), the aqueous phase was extracted with CH₂Cl₂ (10 mL), and the organic extracts were washed with HCl solution (10 mL, 0.5N in H₂O) and NaCl solution (10 mL, sat. aq.), dried over Na₂SO₄, and filtered. Concentration under reduced pressure afforded 2-dihydroxy-3,5-bis(trifluoromethyl)benzimidoyl chloride (308 mg, crude) as yellow oil which was directly used in the next step omitting any intermediate storage.

Step 8: To a solution of 2-dihydroxy-3,5-bis(trifluoromethyl)benzimidoyl chloride (308 mg, 1.0 mmol) in CH₂Cl₂ (10 mL) at 0° C. and under a N₂ atmosphere was added a solution of 2-[1-(2-pyrimidin-2-yl-1,2,4-triazol-3-yl)ethylamino]ethyl benzoate (338 mg, 1.0 mmol) and iPr₂NEt (142 mg, 1.1 mmol), and the resulting reaction mixture was stirred between 0-15° C. for 12 h. Then the mixture was poured into H₂O (20 mL), the aqueous phase was extracted with CH₂Cl₂ (2×20 mL), and the combined organic extracts were washed with HCl solution (20 mL, 0.5N in H₂O) and NaCl solution (20 mL, sat. aq.), dried over Na₂SO₄, filtered, and concentrated under reduced pressure to afford 2-[[N-hydroxy-C-[2-hydroxy-3,5-bis(trifluoromethyl)phenyl]carbon-imidoyl-]-[1-(2-pyrimidin-2-yl-1,2,4-triazol-3-yl)ethyl]amino]ethyl benzoate (400 mg, crude) as yellow oil.

Step 9: To a solution of 2-[[N-hydroxy-C-[2-hydroxy-3,5-bis(trifluoromethyl)phenyl]carbon-imidoyl-1-(2-pyrimidin-2-yl-1,2,4-triazol-3-yl)ethyl]amino]ethyl benzoate (400 mg, 0.657 mmol) in THF (10 mL) at 15° C. under a N₂ atmosphere was added 1,1′-carbonyldiimidazole (213 mg, 1.31 mmol), and the resulting reaction mixture was stirred at that temperature for 12 h. The mixture was poured into H₂O (20 mL), the aqueous phase was extracted with EtOAc (2×20 mL), and the combined organic extracts were washed with NaCl solution (20 mL), dried over Na₂SO₄, filtered and concentrated. Purification by column chromatograph on silica gel (THF/PE =0:100 to 50:50, gradient) afforded 2-[[5,7-bis(trifluoromethyl)-1,2-benzoxazol-3-yl]-[1-(2-pyrimidin-2-yl-1,2,4-triazol-3-yl)ethyl]amino]ethyl benzoate [I-39] (80 mg, 20%) as a yellow solid.

¹H-NMR (400 MHz, CDCl₃, RT): δ ppm 8.71 (br d, J=4.63Hz, 2H), 8.06 (s, 1H), 7.89-7.93 (m, 2H), 7.66 (s, 1H), 7.49-7.55 (m, 2H), 7.34-7.39 (m, 2H), 7.30 (t, J=4.82Hz, 1H), 6.75 (q, J=7.09Hz, 1H), 4.53 (t, J=5.88Hz, 2H), 4.06-4.16 (m, 2H), 1.91 (d, J=7.13Hz, 3H).

### Example 13: Preparation of 2-[[5,7-bis(trifluoromethyl)-1,2-benzoxazol-3-yl]-[1-(2-pyrimidin-2-yl-1,2,4-triazol-3-yl)ethyl]amino]ethanol [I-40]

Step 1: To a solution of 2-[[5,7-bis(trifluoromethyl)-1,2-benzoxazol-3-yl]-[1-(2-pyrimidin-2-yl-1,2,4-triazol-3-yl)ethyl]amino]ethyl benzoate [I-39] (110 mg, 0.186 mmol; cf. Example 12) in MeOH (5 mL) at 15° C. was added K₂CO₃ (51 mg, 0.372 mmol) and the resulting reaction mixture was stirred at that temperature for 3 h. Then the mixture was poured into NH₄Cl solution (10 mL, sat. aq.), the aqueous phase was extracted with EtOAc (2×15 mL), and the combined organic extracts were washed with NaCl solution (20 mL, sat. aq.), dried over Na₂SO₄, filtered, and concentrated under reduced pressure. The residue was purified by preparative HPLC (neutral, MeCN-H₂O) to give 2-[[5,7-bis(trifluoromethyl)-1,2-benzoxazol-3-yl]-[1-(2-pyrimidin-2-yl-1,2,4-triazol-3-yl)ethyl]amino]ethanol [I-40] (80 mg, 44%) as a yellow solid.

¹H-NMR (400 MHz, CDCl₃, RT): δ ppm 8.91 (d, J=4.88Hz, 1H), 8.88-8.93 (m, 1H), 8.20 (br s, 1H), 8.06 (s, 1H), 7.75 (d, J=1.38Hz, 1H), 7.42 (t, J=4.88Hz, 1H), 7.36 (s, 1H), 6.70 (q, J=7.13Hz, 1H), 4.60 (t, J=7.94Hz, 2H), 3.84-4.18 (m, 2H), 1.72 (d, J=7.13Hz, 3H).

### Example 14: Preparation of N-(2-methoxyethyl)-N-[1-(2-pyrimidin-2-yl-1,2,4-triazol-3-yl)ethyl]-5,7-bis(trifluoromethyl)-1,2-benzoxazol-3-amine [I-41]

Step 1: To a solution of 2-[[5,7-bis(trifluoromethyl)-1,2-benzoxazol-3-yl]-[1-(2-pyrimidin-2-yl-1,2,4-triazol-3-yl)ethyl]amino]ethanol [I-40] (100 mg, 0.205 mmol, cf. Example 13) in MeCN (1.5 mL) at 15° C. was added Ag₂O (118 mg, 0.512 mmol) and Mel (58 mg, 0.41 mmol), and the resulting reaction mixture was stirred at that temperature for 12 h. Then the mixture was filtered, and the filtrate was concentrated and purified by preparative TLC (EtOAc=100%) to give N-(2-methoxyethyl)-N-[1-(2-pyrimidin-2-yl-1,2,4-triazol-3-yl)ethyl]-5,7-bis(trifluoromethyl)-1,2-benzoxazol-3-amine [I-41] (60 mg, 58%) as a yellow solid.

¹H-NMR (400 MHz, CDCl₃, RT): δ ppm 8.85 (d, J=4.88Hz, 2H), 8.05 (s, 1H), 7.32-7.39 (m, 3H), 6.48 (q, J=7.09Hz, 1H), 4.42 (t, J=7.69Hz, 2H), 4.07 (q, J=8.17Hz, 1H), 3.80 (q, J=7.42Hz, 1H), 3.59 (s, 3H), 1.79 (d, J=7.13Hz, 3H).

### Example 15: tert-butyl 2-[[5,7-bis(trifluoromethyl)-1,2-benzoxazol-3-yl]-methoxycarbonyl-amino]acetate

Step 1: To a solution of 5,7-bis(trifluoromethyl)-1,2-benzoxazol-3-amine (1.50 g, 5.55 mmol) in THF (15 mL) at 0° C. under a N₂ atmosphere was added potassium bis(trimethylsilyl)amide (1.0M in THF, 11.1 mL, 11.1 mmol), followed after 0.5 h by the dropwise addition of methyl chloroformate (1.57 g, 16.7 mmol), and the resulting reaction mixture was stirred between 0-25° C. for 12 h. Then TLC analysis (PE/EtOAc=5:1) showed the reaction was complete. The reaction mixture was poured into H₂O (50 mL), the aqueous phase was extracted with EtOAc (2×50 mL), and the combined organic extracts were washed with NaCl solution (sat. aq.), dried over Na₂SO₄, filtrated, and concentrated under reduced pressure. Purification by column chromatography on silica gel (EtOAc/PE=0:100 to 15:85, gradient) afforded methyl N-[5,7-bis(trifluoromethyl)-1,2-benzoxazol-3-yl]-N-methoxycarbonyl-carbamate (1.4 g, 65%) as a yellow solid.

¹H-NMR (400 MHz, CDCl₃, RT): δ ppm 8.91 (s, 1H), 8.12 (s, 1H), 8.08 (s, 1H), 8.06 (s, 1H), 7.70 (br s, 1H), 3.94 (s, 3H), 3.87 (s, 6H).

Step 2: To a solution of methyl N-[5,7-bis(trifluoromethyl)-1,2-benzoxazol-3-yl]-N-methoxycarbonyl-carbamate (1.0 g, 2.6 mmol) in MeCN (30 mL) at ambient temperature under a N₂ atmosphere was added tert-butyl 2-bromoacetate (1.52 g, 7.77 mmol) and K₂CO₃ (1.07 g, 7.77 mmol), before the mixture was heated to 80° C. and stirred at that temperature for 16 h. Then TLC (PE/EtOAc=5:1) showed the reaction was completed. The reaction mixture was poured into H₂O (50 mL), the aqueous phase was extracted with EtOAc (2×30 mL), and the combined organic extracts were washed with NaCl solution (sat. aq.), dried over Na₂SO₄, filtrated, and concentrated under reduced pressure. Purification by column chromatograph on silica gel (EtOAc/PE 0:100 to 10:90, gradient) afforded tert-butyl 2-[[5,7-bis(trifluoromethyl)-1,2-benzoxazol-3-yl]-methoxycarbonyl-amino]acetate (1.2 g) as white solid.

¹H-NMR (400 MHz, CDCl₃, RT): δ ppm 8.52 (br s, 1H), 8.04 (s, 1H), 4.61 (s, 2H), 3.94 (s, 3H), 1.48 (s, 9H).

The above tert-butyl 2-[[5,7-bis(trifluoromethyl)-1,2-benzoxazol-3-yl]-methoxycarbonyl-amino]-acetate could, after acidic Boc-cleavage, then be further elaborated into e.g. compound 1-35 analogously as described in Example 11 above.

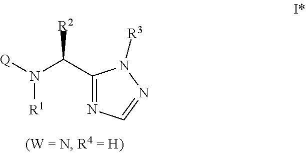

TABLE 1
(the dotted line in R³ denotes the bond to the core structure of formula I*)

| | | | | | | | | phys. data | |
|---|---|---|---|---|---|---|---|---|---|
| | | | | | | | | [HPLC | |
| | | | | | | | | Rt [min], | |
| | | | | | | | | M + H | HPLC |
| No. | R¹ | R² | R³ | Q | R⁵¹ | R⁵² | R⁵³ | [m/z]] | method |
| I-1 | H | CH₃ | 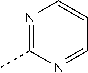 | Q1 | CF₃ | CF₃ | — | 1.14, 443.7 | A |
| I-2 | H | H | 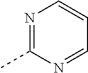 | Q1 | CF₃ | CF₃ | — | 1.14, 429.9 | A |
| I-3 | H | CH₃ | 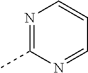 | Q2 | CF₃ | — | — | 0.93, 377.0 | A |
| I-4 | H | CH₃ | 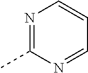 | Q3 | H | CF₃ | CH₃ | 1.01, 388.7 | A |
| I-5 | H | CH₃ | 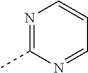 | Q4 | Cl | Cl | — | 1.16, 391.7 | A |
| I-6 | CH₂—c-C₃H₅ | CH₃ | 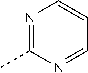 | Q5 | Cl | CF₃ | — | 1.05, 463.8 | A |
| I-7 | H | CH₃ | 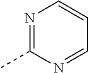 | Q10 | CH₃ | CF₃ | C₂F₅ | 1.06, 456.8 | A |
| I-8 | H | CH₃ | 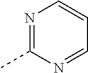 | Q19 | Cl | Cl | — | 0.82, 407.7 | A |
| I-9 | H | CH₃ | 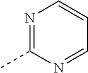 | Q7 | Cl | Cl | — | 0.89, 423.7 | A |
| I-10 | H | CH₃ | 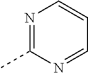 | Q1 | CF₃ | H | — | 0.99, 376.0 | A |
| I-11 | CH₂—c-C₃H₅ | CH₃ | 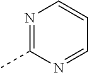 | Q5 | CF₃ | Cl | — | 1.05, 463.9 | A |
| I-12 | H | CH₃ | 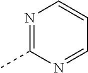 | Q5 | Cl | CF₃ | — | 0.88, 409.9 | A |
| I-13 | H | CH₃ | 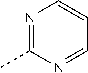 | Q5 | CF₃ | Cl | — | 0.86, 409.8 | A |
| I-14 | H | CH₃ | 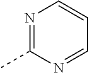 | Q20 | CF₃ | H | — | 0.88, 325.7 | A |
| I-15 | H | CH₃ | 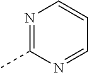 | Q1 | H | CF₃ | — | 0.99, 376.0 | A |
| I-16 | H | CH₃ | 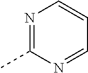 | Q3 | Cl | Cl | CH₃ | 1.09, 389.0 | A |
| I-17 | H | CH₃ | 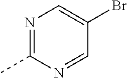 | Q1 | CF₃ | CF₃ | — | 1.28, 523.9 | A |
| I-18 | H | CH₃ | 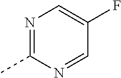 | Q1 | CF₃ | CF₃ | — | 1.21, 375.0 | A |
| I-19 | H | CH₃ | 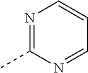 | Q6 | Br | — | — | 0.84, 388.8 | A |
| I-20 | CH₂CH₂—OCH₃ | CH₃ | 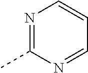 | Q1 | CF₃ | CF₃ | — | 1.21, 501.9 | A |
| I-21 | H | CH₃ | 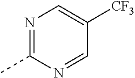 | Q1 | CF₃ | CF₃ | — | 1.31, 511.9 | A |
| I-22 | H | CH₃ | 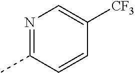 | Q1 | CF₃ | CF₃ | — | 1.42, 510.9 | A |
| I-23 | H | CH₃ | 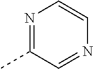 | Q1 | CF₃ | CF₃ | — | 1.25, 443.9 | A |
| I-24 | H | CH₃ | 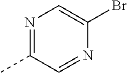 | Q1 | CF₃ | CF₃ | — | 1.37, 523.8 | A |
| I-25 | H | CH₃ | 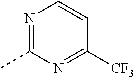 | Q1 | CF₃ | CF₃ | — | 1.30, 511.9 | A |
| I-26 | H | CH₃ | 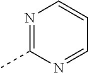 | Q8 | Br | CF₃ | — | 0.95, 454.8 | A |
| I-27 | H | CH₃ | 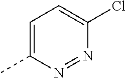 | Q1 | CF₃ | CF₃ | — | 1.30, 477.9 | A |
| I-28 | H | C₂H₅ | 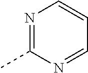 | Q1 | CF₃ | CF₃ | — | 1.23, 457.9 | A |
| I-29 | H | CH₃ | 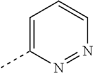 | Q1 | CF₃ | CF₃ | — | 1.19, 443.9 | A |
| I-30 | H | CH₃ | 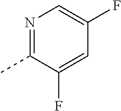 | Q1 | CF₃ | CF₃ | — | 1.26, 478.8 | A |
| I-31 | H | CH₃ | 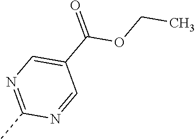 | Q1 | CF₃ | CF₃ | — | 1.30, 516.0 | A |
| I-32 | H | CH₃ | 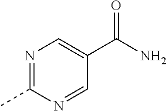 | Q1 | CF₃ | CF₃ | — | 1.06, 487.0 | A |
| I-33 | H | CH₃ | 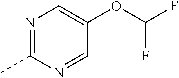 | Q1 | CF₃ | CF₃ | — | 1.25, 509.9 | A |
| I-34 | H | CH₃ | 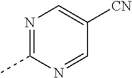 | Q1 | CF₃ | CF₃ | — | 1.20, 468.9 | A |
| I-35 | C(O)OCH₃ | H | 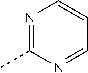 | Q1 | CF₃ | CF₃ | — | 1.21, 487.9 | A |
| I-36 | C(O)OCH₃ | CH₃ | 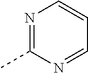 | Q1 | CF₃ | CF₃ | — | 1.22, 501.9 | A |
| I-37 | C(O)OCH₂—c-C₃H₅ | CH₃ | 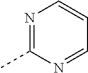 | Q1 | CF₃ | CF₃ | — | 1.32, 542.0 | A |
| I-38 | C(O)OCH₂—c-C₃H₅ | H | 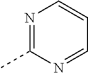 | Q1 | CF₃ | CF₃ | — | 1.32, 528.0 | A |
| I-39 | CH₂CH₂—OC(O)Ph | CH₃ | 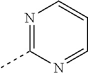 | Q1 | CF₃ | CF₃ | — | 1.38, 592.0 | A |
| I-40 | CH₂CH₂OH | CH₃ | 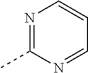 | Q1 | CF₃ | CF₃ | — | 1.23, 488.0 | A |

Biological Examples

If not otherwise specified, the test solutions were prepared as follows:

The active compound was dissolved at the desired concentration in a mixture of 1:1 (vol:vol) distilled water:acteone. The test solution was prepared at the day of use.

B.1. Action on Yellow Fever Mosquito (Aedes aegypti)

For evaluating control of yellow fever mosquito (Aedes aegypti) the test unit consisted of 96-well-microtiter plates containing 200 μl of tap water per well and 5-15 freshly hatched A. aegypti larvae.

The active compounds were formulated using a solution containing 75% (v/v) water and 25% (v/v) DMSO. Different concentrations of formulated compounds or mixtures were sprayed onto the insect diet at 2.5 μl, using a custom-built micro atomizer, at two replications.

After application, microtiter plates were incubated at 28±1° C., 80±5% RH for 2 days. Larval mortality was then visually assessed.

In this test, compounds, I-1, I-2, I-4, I-5, I-10, I-12, I-13, I-15, I-16, I-17, I-18, I-22, I-23, I-24, I-25, I-26, I-27, I-30, I-32, I-33, I-35, I-36, I-37, and I-38, resp. at 2500 ppm showed at least 50% mortality in comparison with untreated controls.

B.2. Boll Weevil (Anthonomus grandis)

For evaluating control of boll weevil (Anthonomus grandis) the test unit consisted of 96-well-microtiter plates containing an insect diet and 5-10 A. grandis eggs. The compounds were formulated using a solution containing 75% v/v water and 25% v/v DMSO. Different concentrations of formulated compounds were sprayed onto the insect diet at 5 μl, using a custom-built micro atomizer, at two replications.

After application, microtiter plates were incubated at about 25±1° C. and about 75±5% relative humidity for 5 days. Egg and larval mortality was then visually assessed. In this test, compound I-1, I-2, I-3, I-4, I-5, I-8, I-9, I-10, I-12, I-13, I-15, I-16, I-17, I-18, I-19, I-21, I-22, I-23, I-24, I-25, I-26, I-27, I-28, I-29, I-30, I-31, I-32, I-33, I-34, I-36, I-37, and I-38, resp., at 2500 ppm showed at least 50% mortality in comparison with untreated controls.

B.3. Tobacco Budworm (Heliothis virescens)

For evaluating control of tobacco budworm (Heliothis virescens) the test unit consisted of 96-well-microtiter plates containing an insect diet and 15-25 H. virescens eggs. The compounds were formulated using a solution containing 75% v/v water and 25% v/v DMSO. Different concentrations of formulated compounds were sprayed onto the insect diet at 10 μl, using a custom-built micro atomizer, at two replications.

After application, microtiter plates were incubated at about 28±1° C. and about 80±5% relative humidity for 5 days. Egg and larval mortality was then visually assessed.

In this test, compounds I-1, I-4, I-5, I-7, I-10, I-15, I-17, I-18, I-21, I-22, I-23, I-25, I-26, I-28, I-29, I-30, I-31, I-32, I-33, I-34, I-36, I-37, and I-38, resp., at 2500ppm showed at least 50% mortality in comparison with untreated controls.

B.4. Green Peach Aphid (Myzus persicae)

For evaluating control of green peach aphid (Myzus persicae) through systemic means the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the aphid diet, using a custom-built pipette, at two replications.

After application, 5-8 adult aphids were placed on the artificial membrane inside the microtiter plate wells. The aphids were then allowed to suck on the treated aphid diet and incubated at about 23 ±1° C. and about 50 ±5% relative humidity for 3 days. Aphid mortality and fecundity was then visually assessed.

In this test, compounds I-1, I-3, I-5, I-6, I-8, I-9, I-10, I-12, I-13, I-15, I-18, I-19, I-25, I-28, I-36, I-37, and I-38, resp., at 2500 ppm showed at least 50% mortality in comparison with untreated controls.

B.5. Cowpea Aphid (Aphis craccivora)

The active compound was dissolved at the desired concentration in a mixture of 1:1 (vol:vol) dis-tilled water: acetone. Surfactant (Kinetic) was added at a rate of 0.01% (vol/vol). The test solution was prepared at the day of use.

Potted cowpea plants were colonized with approximately 30-50 aphids of various stages by manually transferring aphid-infested leaf clippings 24 hours before application. Plants were sprayed with the test solutions using a DeVilbiss® hand atomizer at 20-30 psi (≈1.38 to 2.07 bar) after the pest population had been checked. Treated plants were maintained on light carts at about 25-26° C. Estimate percent mortality was assessed after 72 hours.

In this test, compounds I-12, I-13 I-15, I-18, I-27, I-36, I-37, I-38, resp., at 300 ppm showed at least 50% mortality in comparison with untreated controls.

B.6. Diamond Back Moth (Plutella xylostella)

The active compound was dissolved at the desired concentration in a mixture of 1:1 (vol:vol) distilled water:acetone. Surfactant (Kinetic) was added at a rate of 0.01% (vol/vol). The test solution was prepared at the day of use. Cabbage leaf discs (60 mm in diameter) were dipped in test solution and air-dried. Treated leaves were placed in petri dishes lined with moistened filter paper and inoculated with ten 3rd instar larvae. Mortality was recorded 72 hours after treatment. Feeding damages were also recorded using a scale of 0-100%.

In this test, compounds I-2, I-7, I-10, I-15, I-17, I-18, I-22, I-23, I-25, I-28, I-29, I-30, I-31, I-33, I-34, I-35, I-36, I-37, and I-38, resp., at 300 ppm showed at least 50% mortality in comparison with untreated controls.

B.7. Orchid Thrips (Dichromothrips corbetti)

Dichromothrips corbetti adults used for bioassay were obtained from a colony maintained continuously under laboratory conditions. For testing purposes, the test compound was diluted in a 1:1 mixture of acetone:water (vol:vol), plus Kinetic at a rate of 0.01% v/v.

Thrips potency of each compound was evaluated by using a floral-immersion technique. Each orchid petal was dipped into treatment solution and allowed to dry in Petri dishes. Treated petals were placed into individual re-sealable plastic along with about 20 adult thrips. All test arenas were held under dark condition and a temperature of about 28° C. for duration of the assay. The percent mortality was recorded 72 hours after treatment.

In this test, compounds I-2, I-7, I-8, I-12, I-13,1-17, I-18, I-28, I-36,1-37, and I-38, resp., at 300 ppm showed at least 50% mortality in comparison with untreated controls.

B.8. Green Soldier Stink Bug (Nezara viridula)

The active compound was dissolved at the desired concentration in a mixture of 1:1 (vol:vol) distilled water:aceteone. Surfactant (Kinetic) was added at a rate of 0.01% (vol/vol). The test solution was prepared at the day of use. Soybean pods were placed in 90×50 mm glass Petri dishes lined with moistened filter paper and inoculated with ten late 3rd instar N. viridula. Using a hand atomizer, an approximately 2 ml solution was sprayed into each Petri dish. Treated set-up was kept at about 25-26° C. and relative humidity of about 65-70%. Percent mortality was recorded after 5 days.

In this test, compounds I-12, I-15, I-17, I-18, I-37, and I-38, resp., at 300ppm showed at least 50% mortality in comparison with untreated controls.

B.9. Rice Green Leafhopper (Nephotettix virescens)

Four to five-week old rice seedlings with cut upper leaf portion were cleaned and washed 24 hours before spraying. The active compounds were formulated in 1:1 acetone:water (vol:vol), and 0.01% vol/vol surfactant (Kinetic) was added. Potted rice seedlings were sprayed with 5-6 ml test solution, air dried, covered with Mylar cages and inoculated with 10 adults. Treated rice plants were kept at about 28-29° C. and relative humidity of about 50-60%. Percent mortality was recorded after 72 hours.

In this test, compounds I-8, I-13, I-18, I-37, resp., at 300ppm showed at least 50% mortality in comparison with untreated controls.

B.10. Southern Armyworm (Spodoptera eridania), 2nd instar larvae

The active compounds were formulated by a Tecan liquid handler in 100% cyclohexanone as a 10′000 ppm solution supplied in tubes. The 10′000 ppm solution was serially diluted in 100% cyclohexanone to make interim solutions. These served as stock solutions for which final dilutions were made by the Tecan in 50% acetone:50% water (v/v) into 10 or 20m1 glass vials. A nonionic surfactant (Kinetic®) was included in the solution at a volume of 0.01% (v/v). The vials were then inserted into an automated electrostatic sprayer equipped with an atomizing nozzle for application to plants/insects. Lima bean plants (variety Sieva) were grown 2 plants to a pot and selected for treatment at the 1st true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. Ten to 11 armyworm larvae were placed into the bag and the bags zipped closed. Test plants were maintained in a growth room at about 25° C. and about 20-40% relative humidity for 4 days, avoiding direct exposure to fluorescent light (14:10 light:dark photo-period) to prevent trapping of heat inside the bags. Mortality and reduced feeding were assessed 4 days after treatment, compared to untreated control plants.

In this test, compounds I-5, I-12, I-13, I-16, I-17, I-18, I-22, I-23, I-24, I-25, I-26, I-30, I-33, I-34, I-36, and I-37, resp., at 300 ppm showed at least 50% mortality in comparison with untreated controls.

1. Compounds A compound of formula I 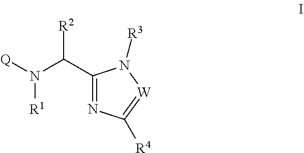
wherein
R¹ is H, OH, C₁-C₆-alkyl, C₁-C₆-haloalkyl, C₃-C₆-cycloalkyl, C₃-C₆-halocycloalkyl, C₁-C₅-alkoxy, C₁-C₄-alkyl-C₃-C₆-cycloalkyl, C₁-C₄-alkyl-C₃-C₆-halocycloalkyl, which groups are unsubstituted, or partially or fully substituted with R¹¹; or C(═N—R¹¹)R¹², C(O)R^{11a};
R¹¹ is CN, C(O)NH₂, C(S)NH₂, CO₂H, NO₂, NR¹²R¹³, OR¹⁴, Si(CH₃)₃; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, 5- or 6-membered hetaryl, or phenyl, which rings are unsubstituted or substituted with halogen, C₁-C₃-haloalkyl, and/or CN;
R₁₁ₐ is C(O)NR¹²R¹³, C(S)NR¹²R¹³, C(O)OR¹⁴, NR¹²R¹³, OR¹⁴, C₁-C₅-alkyl, C₁-C₅-haloalkyl; C₂-C₅-alkenyl; C₂-C₅-haloalkenyl; C₂-C₅-alkynyl; C₂-C₅-haloalkynyl; C₁-C₄-alkoxy-C₁-C₂-alkyl; C₃-C₄-cycloalkyl-C₁-C₂-alkyl, which ring is unsubstituted or substituted with 1 or 2 halogen; 3- to 6-membered heterocyclyl which rings are unsubstituted or substituted with halogen, C₁-C₃-haloalkyl, and/or CN;
R¹², R¹³ are independently from each other H, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl;
m is 0, 1, or 2;
R¹⁴ is H, C₁-C₄-alkyl, C₁-C₄-haloalkyl, C₃-C₆-cycloalkyl, C₃-C₆-halo¬cycloalkyl, C₃-C₄-cycloalkyl-C₁-C₂-alkyl, C₃-C₄-halocycloalkyl-C₁-C₂-alkyl, C(O)—C₁-C₄-alkyl, C(O)—C₁-C₄-haloalkyl, C(O)—C₃-C₄-cycloalkyl, C(O)—C₃-C₄-halo¬cyclo¬alkyl;
R² is H, CN, C₁-C₃-alkyl, C₁-C₃-haloalkyl, C₂-C₃-alkynyl;
R³ is pyridine, pyrimidine, pyrazine, or pyridazine, which rings are unsubstituted or substituted with (R¹¹)ₙ and/or 1 to 3 halogen;
n is 0, 1, 2, or 3;
W is N or C—R⁴;
with the proviso that W is not C—R⁴, if R³ is pyridine;
R⁴ are independently from each other H, halogen, OH, CN, C₁-C₄-haloalkyl, C₂-C₄-alkenyl, C₂-C₄-haloalkenyl, C₂-C₄-alkynyl, C₁-C₄-alkoxy, C₁-C₄-haloalkoxy, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl;
Q is a 5- to 10-membered heteroaryl comprising as ring members 1 to 4 heteroatoms selected from N, O and S which may be oxidized, wherein at least one ring member heteroatom is N, which heteroaryl is unsubstituted, or partially or fully substituted with groups independently selected from R⁵;
R⁵ halogen, OH, CN, SF₅, COOH, CONH₂, NO₂, or C₁-C₆-alkyl, C₃-C₆-cycloalkyl, C₃-C₆-cycloalkyl-C₁-C₆-alkyl, C₁-C₃-haloalkyl, C₁-C₄-alkoxy, C₁-C₃-haloalkoxy, S(O)ₘ—C₁-C₆-alkyl, S(O)ₘ—C₃-C₆-cycloalkyl, S(O)ₘ—C₁-C₃-haloalkyl, S(O)ₘ—phenyl, NR¹²R¹³, NR¹²CO—C₁-C₄-alkyl, NHCO-phenyl, CO₂—C₁-C₄-alkyl, CONR¹²R¹³, CONR¹²(C₃-C₆-cycloalkyl), C(═NO—C₁-C₄-alkyl)R¹²; phenyl and 5- to 6-membered heteroaryl, wherein aromatic rings are unsubstituted, or substituted with 1 to 2 halogen and/or CN; R⁵ groups being unsubstituted, or partially or fully substituted with R¹¹;
two R⁵ present on the same carbon atom may together form a group ═O, ═S, ═NH, ═N(C₁-C₆-alkyl), ═NO(C₁-C₆-alkyl), ═NN(H)(C₁-C₆-alkyl), or ═NN(C₁-C₆-alkyl)₂;
and the N-oxides, stereoisomers, and agriculturally or veterinarily acceptable salts thereof.

2. The compound of formula I according to claim 1, wherein
R¹ is H, OH, C₁-C₂-alkyl, c-C₃H₅CH₂, C₁-C₄-alkoxy-C₁-C₂-alkyl, C(═O)R^{11a} with R^{11a} being c-C₃H₅CH₂ or C₁-C₄-alkoxy.

3. The compound of formula I according to claim 1, which corresponds to formula I.N.

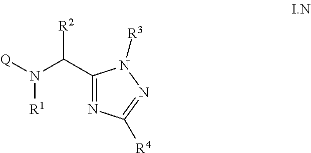

4. The compound of formula I according to claim 1, which corresponds to formula I.1

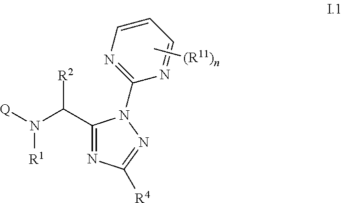

5. The compound of formula I according to claim 1, which corresponds to formula I.2 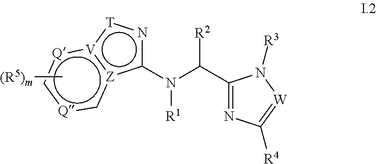
wherein
T is CH, CR⁵, N, O, or S which may be oxidized;
V is C or N;
Z is C or N;
Q′ is CH, CR⁵, or N; and
Q″ is CH, CR⁵, or N;
with the proviso that 2 or 3 of T, V, Z, Q′, and Q″ are a heteroatom.

6. The compound of formula I according to claim 1, to wherein Q is a 5-membered hetaryl containing at least one N as ring member, which ring is partially or fully substituted with R⁵.

7. The compound of formula I according to claim 1, to wherein Q is a 6-membered hetaryl containing at least one N as ring member, which ring is partially or fully substituted with R⁵.

8. The compound of formula I according to claim 1, to wherein Q is selected from the group consisting of Q1 to Q18 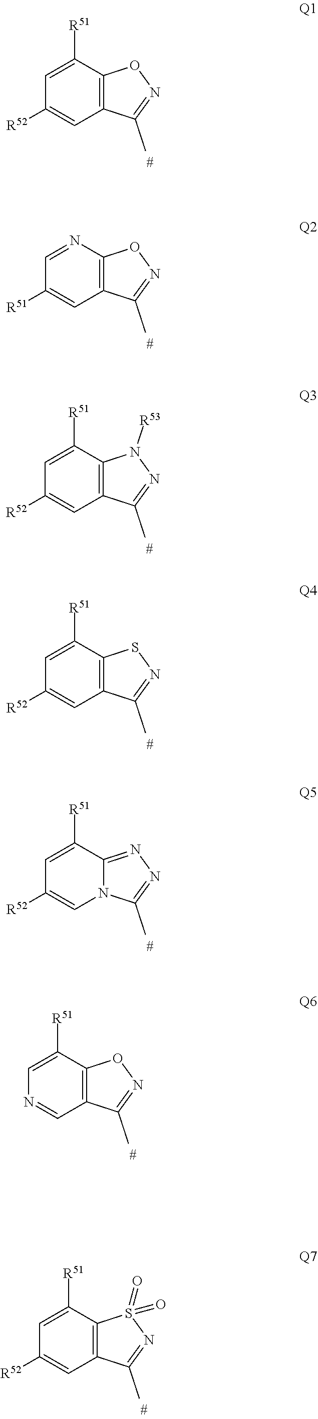 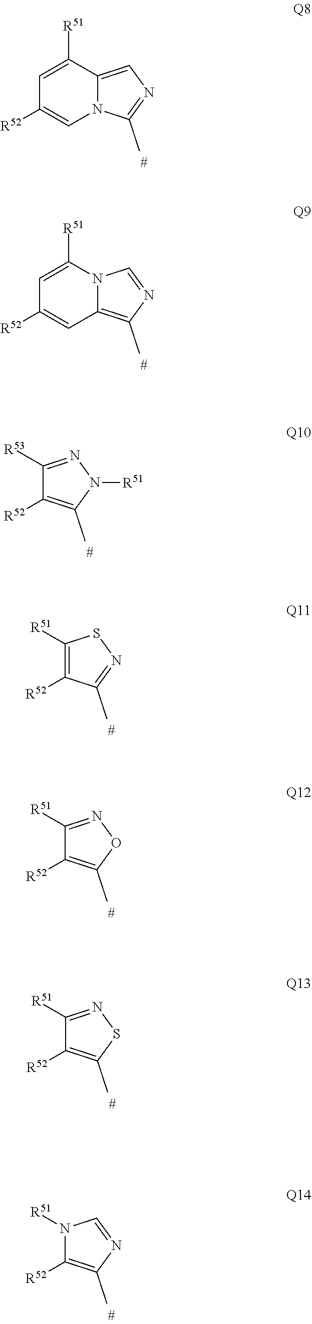 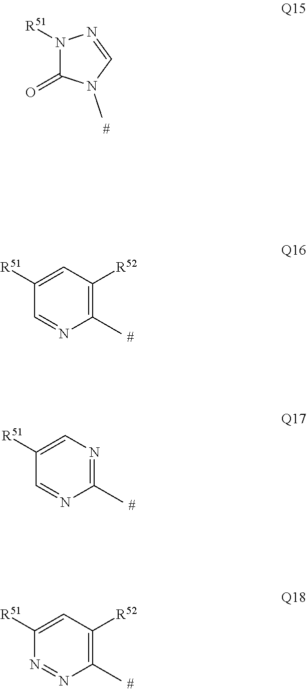
wherein R⁵¹, R⁵², R⁵³ are independently from each other H or R⁵; and
# is the bond to the remainder of the molecule.

9. The compound of formula I according to claim 1, which consist consists mainly of the isomer I.A.

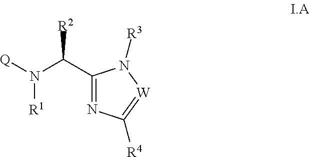

10. An agricultural or veterinary composition comprising at least one compound according to claim 1 and/or at least one agriculturally or veterinarily acceptable salt thereof, and at least one inert liquid and/or solid agriculturally or veterinarily acceptable carrier.

11. An agricultural composition for combating animal pests comprising at least one compound as defined in claim 1 and at least one inert liquid and/or solid acceptable carrier and, optionally, at least one surfactant.

12. A method for combating or controlling invertebrate pests, comprising contacting said pest or its food supply, habitat or breeding grounds with a pesticidally effective amount of at least one compound as defined in claim 1.

13. A method for protecting growing plants from attack or infestation by invertebrate pests, comprising contacting a plant, or soil or water in which the plant is growing, with a pesticidally effective amount of at least one compound as defined in claim 1.

14. A seed comprising a compound as defined in claim 1, or the enantiomers, diastereomers or salts thereof, in an amount of from 0.1 g to 10 kg per 100 kg of seed.

15. A method for treating or protecting an animal from infestation or infection by invertebrate pests comprising bringing the animal in contact with a pesticidally effective amount of at least one compound of the formula I as defined in claim 1, a stereoisomer thereof and/or at least one veterinarily acceptable salt thereof.
